(19) United States
(12) Patent Application Publication
(10) Pub. No.: US 2025/0035619 A1
(43) Pub. Date: Jan. 30, 2025

(54) PROTEIN CORONA BIOMARKER ANALYSIS

(71) Applicant: THE ROYAL COLLEGE OF SURGEONS IN IRELAND, Dublin (IE)

(72) Inventors: Marco Monopoli, Dublin (IE); Duong Trinh Ngoc, Dublin (IE)

(73) Assignee: THE ROYAL COLLEGE OF SURGEONS IN IRELAND, Dublin (IE)

(21) Appl. No.: 18/715,567

(22) PCT Filed: Dec. 5, 2022
(86) PCT No.: PCT/EP22/84435

§ 371 Date: May 31, 2024

(30) Foreign Application Priority Data
Dec. 3, 2021 (GB) 2117557.5

(51) Int. Cl. G01N 33/543; G01N 33/552; G01N 33/574; G01N 33/68

(52) U.S. Cl. CPC G01N 33/54313; G01N 33/552; G01N 33/57423; G01N 33/6884; G01N 2800/52

(57) ABSTRACT

The invention related to a method for selective enrichment of glycoproteins from a sample comprising proteins, the method comprising: —determining the concentration of proteins in the sample or providing the sample with a defined concentration of proteins; incubating particles in the sample to form a protein corona comprising glycoproteins bound to the surface of the particles, wherein the protein concentration to the total surface area of the particles is selected, and/or the particle material is selected, in order to enrich for a specific glycoprotein species on the protein corona; and optionally isolating the protein corona from the sample; and associated methods of screening for biomarkers and diagnosis, and associated compositions.

This invention relates to a method for enriching glycoproteins from a sample, and their use in glycoprotein and glycan profiling, and the diagnosis of diseases or conditions associated with a change in glycoprotein and glycan profile.

N-glycosylation is one of the most important and intricate post-translational modifications of proteins, with respect to the complexity of the added carbohydrates and the magnitude of the cellular machinery devoted to synthesis and modulation (1). In spite of the heterogeneity and diversity, N-glycosylation could be very cell specific and differs significantly in cancer cells (2). Due to the evidence that N-glycan features are altered in many diseases, including different types of cancer, along with the advances of high-throughput characterization techniques, N-glycosylation of plasma proteins has been studied intensively to discover new biomarkers for risk stratification, diagnosis, and prognosis (3-5).

When in contact with biological fluids, the surface of particles, such as nanoparticles (NPs), is spontaneously covered by a selected group of biomolecules including metabolites, lipids and especially proteins, to form a so-called ‘biomolecular corona’ (6). The formation of this layer of biomolecules is ubiquitous in the medicine context, depending on the properties of both NPs and biological fluids. Biomolecular corona holds the potential to contribute significantly to the biomarker discovery field, thanks to the unique set of molecules bound to the surface of specific NPs (7). Previous studies have shown that the protein corona obtained from different biological fluids could distinguish the healthy individuals from people with disease states (8-10). From a common viewpoint of proteomic and glycoproteomic analyses, the formation of protein corona can be exploited as an enrichment step that provides specific sets of proteins from a complex sample, like human plasma.

Despite being a convenient and information-rich sample for biomarker discovery, plasma has a highly complex protein composition, containing more than 10,000 proteins, whose concentrations range more than 10 orders of magnitude. Furthermore, top 12 abundant proteins, including albumin, immunoglobulins and transferrin, account for more than 90% of total plasma protein mass (11-13). Meanwhile, a typical upper limit of dynamic range in commercial mass spectrometry (MS) is much lower, about 5 orders of magnitude, which leads to inevitable information loss in low abundant proteins (14). The presence of glycosylation adds another layer of complexity to the plasma analysis as the N-glycosylation profiling can face different problems, mainly coelution of glycan structures and MS glycan ion suppression, which likely hinders the detection and quantification of potential biomarker traits (15). As a result, different enrichment strategies have been developed to reduce the plasma complexity, including fractionation, immunoaffinity chromatography and glycoprotein enrichment (lectin affinity, hydrazide and HILIC chromatography); each with their own advantages and drawbacks (16, 17). The relative abundance of plasma proteins can be very different from those of plasma N-glycoforms. While plasma proteins with very low abundances have been the main target of proteomic biomarker discovery, glycan profiles of fairly abundant glycoproteins could also provide useful information about the disease state. In fact, many glycan-based biomarker studies have focused on the glycosylation of immunoglobulins and acute phase proteins, including haptoglobin, transferrin and alpha-1-acid glycoprotein (18). These proteins were mainly separated from plasma using immunoaffinity chromatography method, which is protein-specific but can be costly for a routine or large-scale cohort analysis.

Although biomolecular corona has emerged as a promising tool for disease stratification, no study has managed to successfully exploit the glycan profiles of corona proteins for this purpose.

An aim of the present invention is to improve the use of biomolecular corona for use in disease stratification.

According to a first aspect of the present invention, there is provided a method for selective enrichment of glycoproteins from a sample comprising proteins, the method comprising:
determining the concentration of proteins in the sample or providing the sample with a defined concentration of proteins;
incubating particles in the sample to form a protein corona comprising glycoproteins bound to the surface of the particles, wherein the protein concentration to the total surface area of the particles is selected, and/or the particle material is selected, in order to enrich for a specific glycoprotein species on the protein corona; and
optionally isolating the protein corona from the sample.

According to another aspect of the present invention, there is provided a method for enriching glycoproteins from a sample comprising proteins, the method comprising:
determining the concentration of proteins in the sample or providing the sample with a defined concentration of proteins;
adding particles suitable for forming a protein corona to the sample, using a ratio of total proteins to the particle total surface specific ranges to isolate specific glycoproteins;
incubating the particles in the sample to form a protein corona comprising glycoproteins bound to the surface of the particles; and
optionally isolating the protein corona from the sample.

The method may further comprise the step of determining the glycoprotein and/or glycan profile of the protein corona isolated from the sample.

Advantageously, the present invention uses particles, such as nanoparticles, as a platform to enrich glycoproteins, such as fibrinogen, in a protein corona. This can be done with ease for samples, such human plasma, where the protein corona's proteomic and glycomic profiles can be determined. A study on a lung cancer cohort identified the use of these proteomic and glycan profiles for the disease diagnosis. The present invention further recognises that although the use of protein corona is known for biomarker profiling, it has not been previously recognised that the glycoprotein content of a sample, which forms a protein corona around a particle is specifically enriched at an optimum particle surface area relative to the protein concentration in the sample. In particular, due to the progressive displacement of glycoproteins such as fibrinogen and Apolipoprotein (Apo) A1 by other particle binding molecules, such as kallikrein B and plasminogen, there is a critical need to control the surface area of the particle relative to the protein concentration in order to elucidate a disease-indicating glycoprotein and/or glycan profile, that would otherwise be hidden in standard samples.

### The Sample

The sample may comprise a matrix of constituents. The sample may comprise a mixture of proteins, such as a complex mixture of proteins. In one embodiment, the sample is a fluid sample. The sample may comprise a bodily fluid sample. In another embodiment, the sample may be a tissue sample. The bodily fluid sample may comprise or consist of blood, blood fractions, plasma, saliva, sweat, lymphatic fluid, Liquid biopsy, cerebral spinal fluid (CSF), synovial fluid, vaginal secretion, urine, faecal matter, mucous, tissue or cell lysate, or any sample of fluid derived from human or animal species or from the environment. In a preferred embodiment, the sample comprises or consist of blood, or a protein-containing fraction thereof. Further preferably, the sample is a plasma sample. Preferably the sample is a bodily fluid sample which comprises glycoprotein.

The bodily fluid sample may be from a mammalian subject, preferably a human. In one embodiment, the sample is from a non-human animal subject, such as livestock or domestic animal (e.g. horse, cow, pig, sheep, dog or cat). The subject may be suffering from a disease or condition, such as cancer. In another embodiment, the subject may be suspected of suffering from a disease or condition, such as cancer. Alternatively, the subject may be a healthy subject (e.g. not diagnosed or suspected to suffer from a disease or condition to be determined by the method herein, such as cancer).

In another embodiment, the sample may comprise a sample of cells or tissue lysate. In another embodiment, the sample may comprise a cell culture sample or used cell media sample. In another embodiment, the sample may comprise an environmental or industrial sample, for example from a water body or stream.

Protein aggregates may be removed prior to incubation with the particles, for example by centrifugation. The protein concentration determination may be following the removal or reduction of any protein aggregates in the sample.

The skilled person will be familiar with methods to determine the concentration of proteins in the sample and/or methods of diluting the sample to a pre-determined protein concentration. For example the protein concentration may be determined by a UV-vis/A280 assay, a Bradford assay or a BCA assay or any other suitable method to determine protein concentration.

The sample may be diluted with an aqueous solution, such as a buffer. In one embodiment, the sample, such as plasma, is diluted with PBS buffer, such as PBS at 10 mM, pH 7.4.

In one embodiment, the sample is diluted or provided at a sufficient concentration to lead to an excess of surface area to the total proteins, such as a particle concentration of 1 mg/ml and a protein range of 0.33 or less to 9.9 mg/ml which corresponds to a ratio of 11 to 330 mg/m², optionally wherein the glycoprotein to be enriched is fibrinogen. In another embodiment, 1 mg/ml of silica nanoparticles exposure to 9.9 to 52.8 mg/ml, which corresponds to a ratio of 660 to 1760 mg/m², may lead to the prefractionation of histidine rich glycoproteins, such as kininogen and/or plasminogen.

In one embodiment, preferably for enrichment of fibrinogen, for 1 mg/ml of particles, such as silica particles, there is a protein concentration of 0.33 to 9.9 mg/ml. In one embodiment, preferably for enrichment of fibrinogen, the protein concentration to total surface area of the particles may be 11 to 330 mg/m².

In another embodiment, preferably for enrichment of histidine rich glycoproteins, such as kininogen and/or plasminogen, for 1 mg/ml of particles, such as silica particles, there is a sample protein concentration of 9.9 to 52.8 mg/ml. In another embodiment, preferably for enrichment of histidine rich glycoproteins, such as kininogen and/or plasminogen, the sample protein concentration to total surface area of the particles may be 660 to 1760 mg/m².

In one embodiment, the sample protein concentration to total surface area of the particles may be about 11 to 1760 mg/m².

The skilled person will recognise that the concentration of particles may be adjusted to achieve the relevant total surface area or the concentration of the protein in the sample may be adjusted, for example by dilution of the sample.

### The Particles

For example, for silica nanoparticles of 100 nm a fibrinogen enrichment occurs with a protein amount of 0.33 to 9.9 mg/ml to every 1 mg/ml of nanoparticles which corresponds to 11 to 330 mg/m², or other proteins (including histidine rich glycoprotein, kininogen or plasminogen) may be prefractionated using a different range within 330-1760 mg/m², which also corresponds to 9.9 mg/ml to 52.8 mg/ml of proteins to 1 mg/ml of nanoparticles.

In one embodiment, every 1 mg/ml of silica nanoparticles in the sample is exposed to 0.33 to 9.9 mg/ml of protein, which may lead to a fibrinogen enrichment.

The protein concentration to total surface area of the particles may be expressed as a ratio, such as Ratio=protein mass/total particle surface area.

In one embodiment, for example to enrich for fibrinogen, the surface area of the particles may be in excess of the protein in the sample (i.e. such that there is more surface area of particle than can be bound by the protein in the sample). In an alternative embodiment, for example to enrich for histidine rich glycoproteins, such as kininogen and plasminogen, the protein in the sample may be in excess of the surface area of the particles. The particles for forming the protein corona may comprise any suitable material which adsorbs protein, preferably glycoprotein, onto its surface and for example leads to a prefractionation from the matrix from which the protein was contained. Preferably the particles for forming the protein corona may comprise any suitable material which adsorbs fibrinogen onto its surface. Such material may be a core material or a coating on another material. In one embodiment, the particles comprise or consist of silica, polystyrene, metal, polymer or carbon particles or core-shell particles (e.g. solid core with a porous shell, such as porous silica shell). The particles may be coated, for example with a polymeric or silica coating on a metal core particle to enable glycoprotein adsorption and magnetic separation. The metal may be iron oxide or gold. The polymeric coating may comprise a copolymer, such as PLGA. In one embodiment, the particles may comprise functional groups on their surface to enhance adsorption or binding to glycoproteins such as fibrinogen. Such functional groups may comprise sulphur, carboxylic acid. In one embodiment, the particles comprise or consist of silica. In one embodiment, the particles comprise or consist of silica coated iron oxide particles.

In one embodiment, the particles are nanoparticles, such as 1-100 nm in size. The particles may be about 1-100 nm in size of at least one dimension or any other particle larger than 100 nm. The particles may be about 1-500 nm in size of at least one dimension, or their widest dimension. In a preferred embodiment, the particles are about 100 nm. The particle size may refer to an average particle size in a population of particles as measured by their widest dimension. In a preferred embodiment, the particle size may be determined by at least one dimension of the particle as an average of a population of particles. For example, carbon nanotubes can be longer than 100 nm but thinner than 100 nm and are still recognised as nanoscale. The particles may be of any shape and surface topography to provide the desired surface area. In one embodiment, the particles a substantially spherical. Additionally or alternatively the particles may have a smooth/planar surface topography. In an embodiment wherein the particles are porous, the pores may be sufficiently small to be impenetrable to glycoprotein, such as fibrinogen.

In one embodiment, the concentration of particles for incubation with the sample is provided at a sufficient ratio to prefractionate selective glycoproteins and a skilled person will recognise that the concentration of particles may be adjusted to achieve the relevant total surface area or the concentration of the protein in the sample may be adjusted, for example by dilution of the sample.

In one embodiment, the particles are incubated with the sample at a concentration of about 1 mg/ml.

The particles may be porous or non-porous. The provision of pores may be provided to increase the particle surface area.

The skilled person will recognise that the surface area of the particles in the sample may be adjusted by changing one or more of the particle concentration, the particle size and the porosity of the particle.

### Incubation

The particles may be incubated in the sample for a period sufficient to enrich glycoproteins, such as fibrinogen from the sample. Preferably the particles may be incubated in the sample for a period sufficient to reach equilibrium with regard to the surface adsorption of glycoproteins, such as fibrinogen from the sample. The particle may be incubated in the sample for a period of at least 2 minutes. In another embodiment, the particle may be incubated in the sample for a period of at least 5 minutes. In another embodiment, the particle may be incubated in the sample for a period of at least 10 minutes. In another embodiment, the particle may be incubated in the sample for a period of at least 20 minutes. In another embodiment, the particle may be incubated in the sample for a period of at least 30 minutes. In another embodiment, the particle may be incubated in the sample for a period of at least 1 hour. In another embodiment, the particle may be incubated in the sample for a period of at about 30 minutes to 24 hours. In another embodiment, the particle may be incubated in the sample for a period of at about 1 hour.

The incubation of the particles in the sample allows for the selective adsorption and displacement of the glycoproteins.

The particles may be incubated in the sample at any temperature that does not result in the denaturing of glycoproteins, such as fibrinogen. The particles may be incubated in the sample at a temperature of 40° C. or less, preferably 37° C. or less. The particles may be incubated in the sample at a temperature of between 15° C. and 37° C. The particles may be incubated in the sample at a temperature of between 25° C. and 37° C. In a preferred embodiment, the particles are incubated in the sample at a temperature of about 37° C.

The particles may be incubated in the sample with agitation, such as stirring.

In a preferred embodiment, the particles may be incubated under suitable conditions and time for enrichment of glycoproteins (e.g. fibrinogen) from the sample, preferably for the glycoprotein (e.g. fibrinogen) to reach an adsorption equilibrium on the surface of the particles. In a preferred embodiment, the particles may be incubated at about 37° C. for 1 hour with continuous agitation.

### Isolation of the Protein Corona

In one embodiment, the protein corona may be analysed for glycoprotein and/or glycan profile directly in the sample, such as plasma. The direct analysis in the sample may comprise direct on plasma analysis.

The protein corona may be isolated from the sample by any appropriate means known to the skilled person, and resuspended into a resuspension solution. In one embodiment, the protein corona is isolated from the sample by centrifugation and resuspension of the protein corona containing fraction/pellet in a resuspension solution. The centrifugation may be ultracentrifugation. The centrifugation may be at 15,000 RCF or more. The centrifugation may be for a period of at least 5, 8 or 10 minutes. The centrifugation may be at about 18,000 RCF for about 10 minutes.

In another embodiment, the protein corona may be isolated from the sample by chromatography, such as size exclusion chromatography, ion exchange chromatography, hydrophobic chromatography, affinity chromatography, or immunoaffinity chromatography. In one embodiment, the protein corona can be isolated from the sample by affinity chromatography, for example wherein the protein corona is prepared with an appropriate affinity tag. In another embodiment, the protein corona can be isolated from the sample by magnetic separation, for example with the use of a ligand-coated metal particle and an affinity tag (arranged to bind the ligand) on the protein corona. Alternatively, the particle used to form the protein corona may itself comprise a metal and be subject to magnetic forces for separation.

The resuspension solution may comprise water, saline or buffer. The resuspension solution may comprise buffer (which may otherwise be termed “resuspension buffer”).

### Washing of the Protein Corona

In one embodiment, the method further comprises performing one or more wash steps following isolation of the protein corona. The number of washes of the protein corona isolated from the sample may be between 1-3. In a preferred embodiment, the isolated protein corona is washed only once. The skilled person will recognise that the initial isolation of the protein corona from the sample may not constitute a wash step.

Advantageously, it has been found that a single wash step can provide sufficient clarity of the enriched glycoprotein content of the protein corona to provide a unique glycan profile, whilst also minimising the potential loss of the glycan profile in subsequent washes.

Washing the protein corona may comprise removing the protein corona from the resuspension solution and re-introducing the protein corona into another resuspension solution. In one embodiment the protein corona are washed by centrifugation into a pellet and resuspension in the resuspension solution.

In another embodiment, washing the protein corona may comprise the use of chromatography, such as size exclusion chromatography, dialysis, or magnetic separation, for example wherein the particles comprise a metallic component.

### Determining the Glycoprotein Profile

In one embodiment, the glycoprotein content of the protein corona is determined. In another embodiment, the glycoprotein content of the protein corona is determined and the glycan profile of the glycoproteins enriched in the protein corona is determined.

The glycoprotein content may be the total glycoproteins isolated per ml of sample or per mg of protein corona. Additionally or alternatively, the relative level of glycoproteins may be determined. In a preferred embodiment, the level and identity of the glycoproteins is determined.

Any suitable determination method may be used by the skilled person to determine the glycoprotein profile of the protein corona. The glycoproteins may be determined by chromatography and/or mass spectrometry. In one embodiment, the glycoprotein content may be determined by shotgun proteomics.

In another embodiment, the glycoprotein content of the protein corona may be determined by immunodetection, affinity chromatography, hydrazide capture, HILIC, or lectin chromatography.

### Determining the Glycan Profile

The glycan profile may be determined by any method known to the skilled person, which is capable of detecting glycan structural changes. Such methods may comprise Raman and Raman optical activity (ROA) spectroscopy, plate-based assays and affinity-based assays such as ELISA. The glycan profile may be determined by glycan release, fluorophore labelling followed by chromatography. Alternatively, HPAE-PAD (High-Performance Anion-Exchange chromatography with Pulsed Amperometric Detection) may be used to analyse the glycan profile. Mass spectrometry may be used for analysis of released glycans. Glycopeptides may proteolyze from glycoproteins with an enzyme, such as trypsin.

In one embodiment, the glycan profile of the glycoproteins enriched in the protein corona is determined. Determining the glycan profile of the protein corona isolated from the sample may comprise the step of analysing the glycan content of enriched glycoproteins on the isolated and washed protein corona. The glycan profile may be the total glycans isolated per ml of sample or per mg of protein corona. Additionally or alternatively, the relative level of glycans may be determined. In a preferred embodiment, the level and identity of the glycans is determined.

Any suitable determination method may be used by the skilled person the determine the glycan profile of the protein corona. In one embodiment, the glycans are separated from their glycoprotein prior to determination, for example by enzyme digestion. The glycoproteins present on the protein corona may be deglycosylated to release N-glycans for analysis. In one embodiment, an enzyme, such as PNGaseF, may be used to cleave the linkage between the core N-Acetylglucosamine (GlcNAc) and the asparagine residue on the glycoproteins, thereby releasing all N-glycans except those containing fucose α1-3 linked to the reducing terminal GlcNAc. The glycans may alternatively be released by chemical release, such as hydrazinolysis or alkylamine release.

The glycans may be determined by liquid chromatography for purification/separation, followed by mass spectrometry to determine the glycan structures. In one embodiment, the glycans released from the glycoproteins of the protein corona are tagged with a fluorescent label. The fluorescently labelled glycans may be analysed by hydrophilic-interaction liquid chromatography (HILIC) with fluorescence detection. In one embodiment, the glycans are labelled by reductive amination, for example with procainamide. Glycan labels may comprise 2-Aminobenzoic acid (2-AA) labeling via reductive amination, 1-phenyl-3-methyl-5-pyrazolone labeling via a Michael-type addition or labeling with phenylhydrazide. In another embodiment the glycans are labelled with 2-amino benzamide (2AB), glycosylamine tags or a procainamide tag (e.g. which is MS compatible).

The chromatography may comprise HILIC-AEX (combined hydrophilic-interaction liquid chromatography and anion exchange chromatography). The mass spectrometry may be electrospray ionization mass spectrometry (ESI-MS).

### Further Aspects

According to another aspect of the present invention there is provided a method of glycoprotein profiling of a sample, wherein the glycoprotein profile of the sample is determined by enrichment of glycoproteins according to the method of the invention.

The method of glycoprotein profiling may further comprise a step of determining the glycan profile of the enriched glycoprotein(s).

According to another aspect of the present invention there is provided a method of glycan profiling of a glycoprotein in a sample, wherein the glycan profile is determined by enrichment of the glycoprotein according to the method of the invention and determining the glycan content of the glycoprotein.

In one embodiment, the glycoprotein comprises or consists of fibrinogen and/or apolipoprotein (Apo) A1. In one embodiment, the method of the invention is used to enrich fibrinogen and/or apolipoprotein (Apo) A1 from a sample for glycan profiling. In another embodiment, the glycoprotein comprises histidine rich glycoproteins, such as kininogen and/or plasminogen.

According to another aspect of the present invention there is provided a method of diagnosis of a disease or condition of a subject, the method comprising the enrichment of glycoproteins in a sample from the subject in accordance with the method of the invention, wherein the glycoprotein and/or glycan profile of the protein corona is determined; and
wherein the glycoprotein and/or glycan profile is indicative of a disease or condition.

According to another aspect of the present invention there is provided a method of monitoring the progression or remission of a disease or condition in a subject, the method comprising the enrichment of glycoproteins in a first sample from the subject in accordance with the method of the invention, wherein the glycoprotein and/or glycan profile of the protein corona is determined and the glycoprotein and/or glycan profile is indicative of the state of the disease or condition; and
further comprising the enrichment of glycoproteins in a subsequent sample from the subject in accordance with the method of the invention, wherein the glycoprotein and/or glycan profile of the protein corona is determined and wherein a change in the determined glycoprotein and/or glycan profile between the first sample and subsequent sample is indicative of the progression or remission of the disease or condition.

The method may comprise three or more glycoprotein and/or glycan profile determination from sequential samples, for example over the course of a treatment. The first sample may be taken from the subject before treatment, and a subsequent sample may be taken after or during treatment. Additionally or alternatively, the samples may be taken at different time points, for example separated by one or more weeks, one or more months, or one or more years.

According to another aspect of the present invention there is provided a method of screening for biomarkers of a disease or condition, the method comprising the comparison of the glycoprotein and/or glycan profile of the protein corona from a sample of one or more subjects afflicted with the disease or condition relative to a control, such as the glycoprotein and/or glycan profile of the protein corona from a sample of one or more subjects that are not afflicted with the disease or condition;
wherein a difference in glycoprotein and/or glycan profile is indicative of a biomarker for the disease or condition.

The methods of the invention may further comprise a treatment step, for example wherein a subject that is diagnosed with a disease or condition in accordance with the invention is further administered with a therapy for the disease or condition or the subject may have surgery to remove afflicted tissue, such as cancerous tissue. In an embodiment wherein the disease or condition is cancer, the therapy may comprise the administration of a cancer treatment, such as chemotherapy and/or immunotherapy. The treatment may comprise gene therapy. In another embodiment, the treatment may comprise a cell transplant, such as a T-cell transplant, and/or other immune cell therapy. In one embodiment, the treatment may comprise the administration of a vaccine. In one embodiment, the treatment may comprise surgery.

According to another aspect of the present invention there is provided a method of treatment for cancer in a subject, the method comprising:
obtaining or having obtained results of a cancer diagnosis for the subject in accordance with the method of the invention herein; and
administering a treatment for the cancer to the subject.

The treatment may comprise one or more of chemotherapy, radiotherapy, immunotherapy or surgery (for example, a lobectomy, pneumonectomy, wedge resection, radiofrequency ablation, cryotherapy or photodynamic therapy).

The method of treatment may further comprise the monitoring of progression of the cancer in accordance with the invention.

According to another aspect of the present invention there is provided a method of diagnosis for lung cancer, the method comprising the detection of one or more lung cancer biomarkers in a sample from a subject, wherein the biomarkers are selected from fibrinogen-derived glycan FA2G2S1 and FA3G3S2;
wherein a change in the level FA2G2S1 and FA3G3S2 is indicative of lung cancer, optionally wherein the lung cancer is non-small cell lung cancer.

The method of diagnosis for lung cancer may comprise the determination of the presence or level of the biomarkers in accordance with the method of the invention herein. In one embodiment, a decrease in the level FA2G2S1 and/or FA3G3S2 is indicative of lung cancer.

### The Disease/Condition

In one embodiment, the disease comprises cancer. In one embodiment, the cancer is lung cancer. The lung cancer may comprise non-small cell lung cancer (NSCLC) or small cell lung cancer. In a preferred embodiment, the cancer is non-small cell lung cancer (NSCLC). The non-small cell lung cancer (NSCLC) may be adenocarcinoma, squamous cell carcinoma or large cell carcinoma. In one embodiment the cancer is adenocarcinoma and/or squamous cell carcinoma. In another embodiment, the cancer may comprise prostate, breast, liver, pancreatic, or ovarian cancer.

In one embodiment, the disease comprises autoimmune or inflammatory diseases, such as rheumatoid arthritis, inflammatory bowel disease, or systemic lupus erythematosus.

### Composition

According to another aspect of the present invention there is provided a composition comprising a sample and a protein corona suspension within the sample, wherein the protein corona is formed around particles, such as silica particles, at a protein to particle surface area ratio of 11-330 mg/m². Such a composition may enrich fibrinogen on the protein corona.

According to another aspect of the present invention there is provided a composition comprising a sample and a protein corona suspension within the sample, wherein the protein corona is formed around particles, such as silica particles, at a protein to particle surface area ratio of 660 to 1760 mg/m². Such a composition may enrich histidine rich glycoproteins on the protein corona.

In a preferred embodiment, the sample is a plasma sample. In one embodiment, the protein corona comprises a glycoprotein species which is enriched from the protein in the sample. The glycoprotein may be fibrinogen and/or apolipoprotein (Apo) A1, or histidine rich glycoproteins.

### Enrichment of Specific Glycoproteins

According to another aspect of the present invention, there is provided a method of identifying a particle for enrichment of a glycoprotein species from a sample comprising proteins, the method comprising:
determining the concentration of proteins in the sample or providing the sample with a defined concentration of proteins;
incubating particles in the sample to form a protein corona comprising glycoproteins bound to the surface of the particles, wherein the most protein corona-enriched glycoprotein of the sample is identified, and wherein the total surface area of the particles relative to the protein concentration, and/or the particle material is selected as suitable for protein corona-enrichment of the identified glycoprotein.

### Definitions

The term “enrichment” used herein is intended to refer to the accumulation of glycoprotein(s) on a particle surface, such that they are sequestered or localised around a protein corona.

The term “glycan profile” may refer to the absolute and/or relative number of the isolated glycans, and the structural identity of some or all of the isolated glycans.

The term “glycoprotein profile” may refer to the absolute and/or relative number of the isolated glycoproteins, and the identity of some or all of the isolated glycoproteins.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

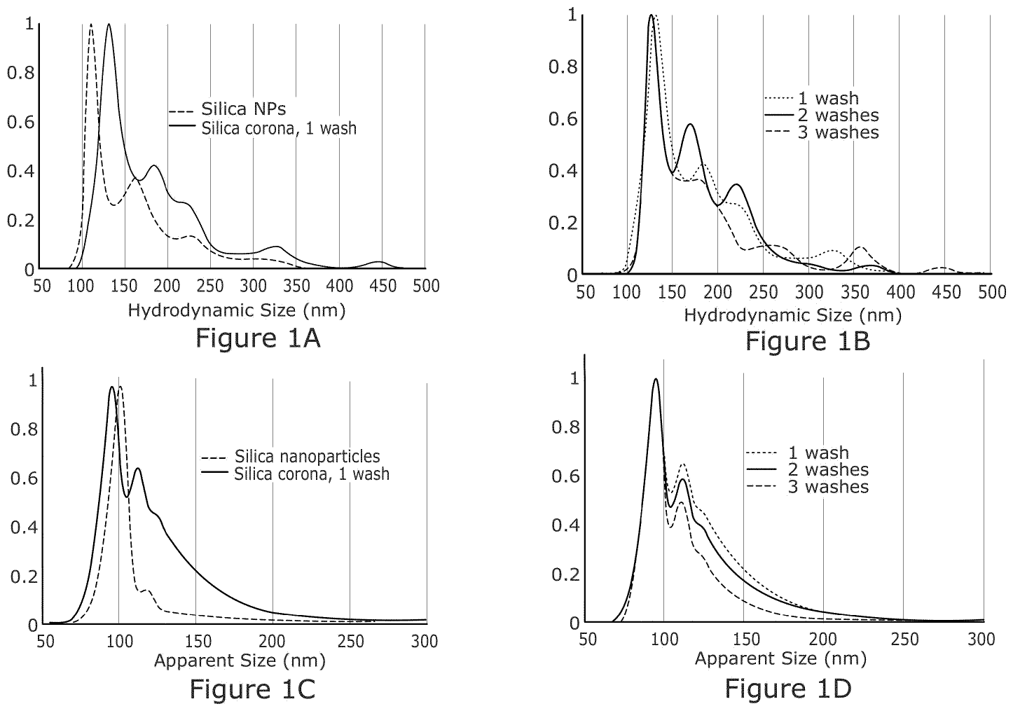

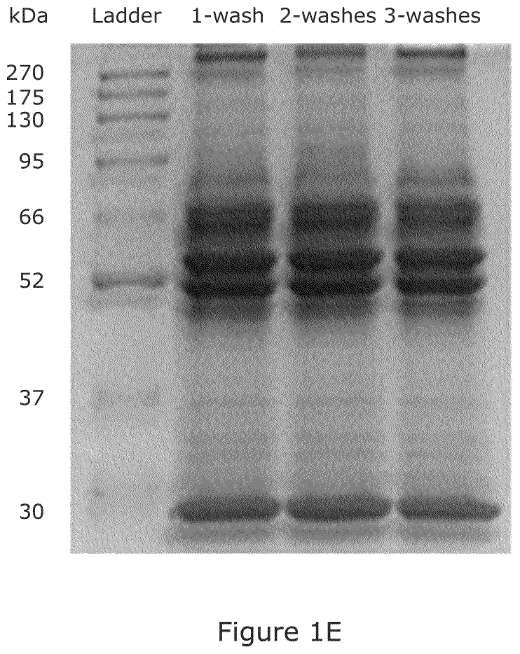

FIG. 1: Characterization of silica corona 3% plasma after 1, 2 and 3 washes. A) Hydrodynamic size distributions of pristine silica NPs and silica corona 3% plasma, 1 wash. B) Hydrodynamic size distributions of silica corona 3%, 1, 2 and 3 washes. The data were shown as the average of 3 measurements by NTA. The peaks were normalised against the tallest peaks (highest particle concentration) that had a value of 1. C) DCS size distributions of pristine silica NPs and silica corona 3% plasma, 1 wash. D) DCS size distributions of silica corona 3%, 1, 2 and 3 washes. The data were shown as relative weight particle size distribution. The tallest peak (highest weight value) had a value of 1 and all other particle size peaks were then normalized against this base peak to give a relative weight distribution.

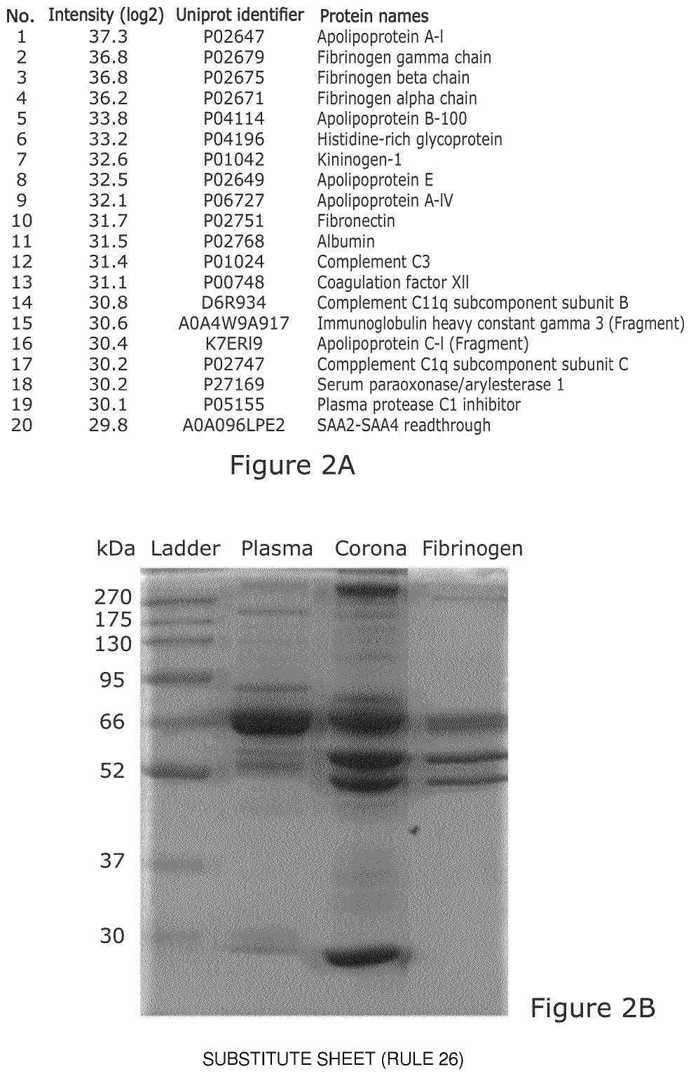

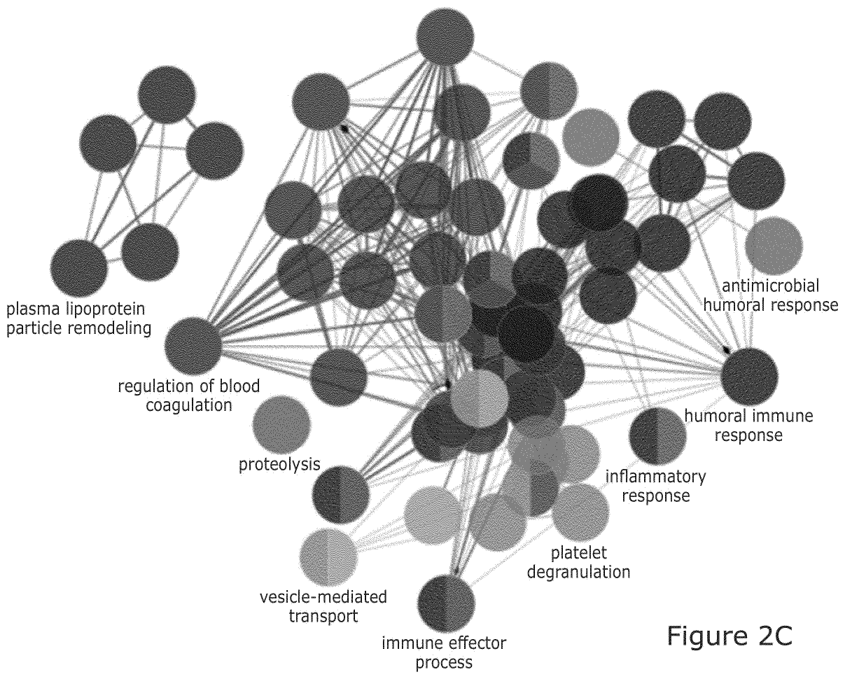

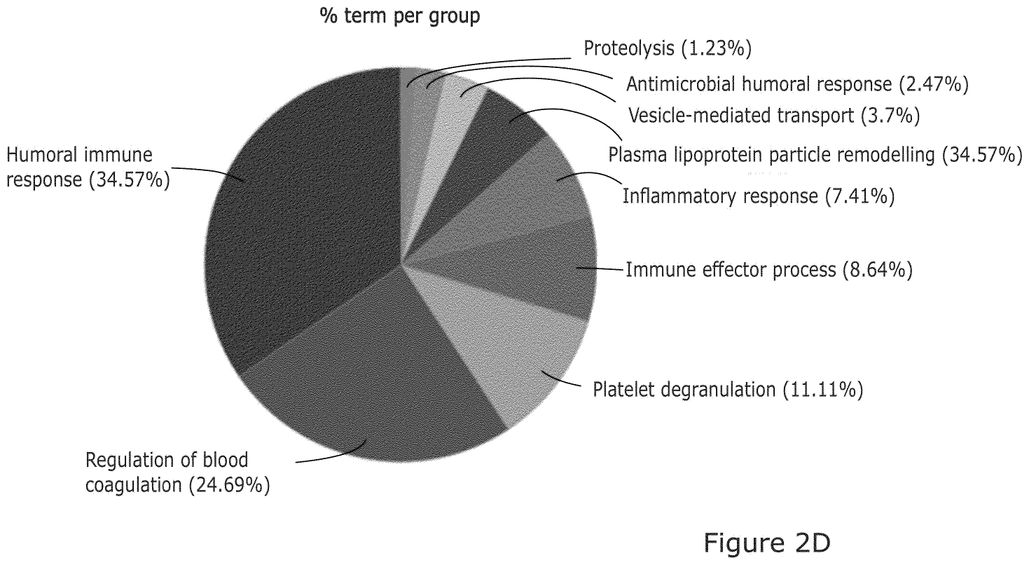

FIG. 2: Proteomic features of silica corona 3% plasma corona. A) Top 20 abundant proteins in the corona, from the total of 291 proteins identified by Mass Spectrometry. B) SDS-PAGE gel of the corona proteins in comparison with full plasma and fibrinogen. C) Directed acyclic graph (DAG) shows the GO enrichment based on the Biological Process term. The analysis was performed with ClueGo/Cytoscape, only pathways with Bonferroni-corrected p-values <10⁻⁶ are shown. D) The term percentages of nine enriched protein biological process groups.

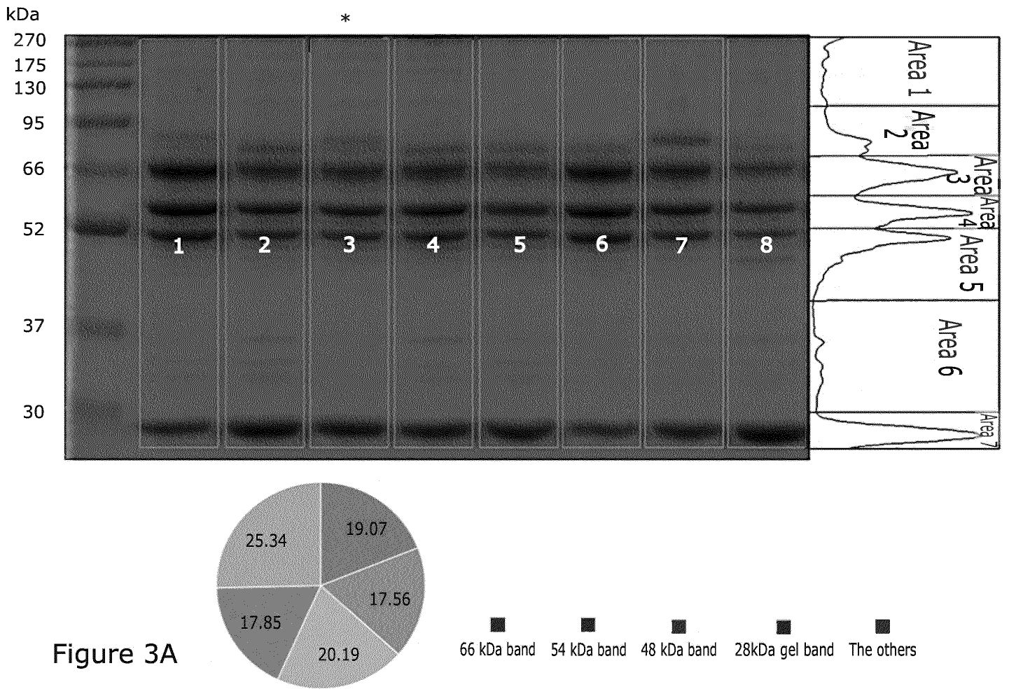

FIG. 3: Gel densitometry in ImageJ and the intensity percentages of proteins bands in the silica corona (A) and full plasma (B). A) Four strong bands were identified at 66, 54, 48 and 28 kDa. B) A strong band of albumin can be seen at 66 kDa. The band intensities normalised by total were shown in the corresponding pie charts. A protein band above 270 kDa marker was omitted from the analysis due to its close proximity to the top gel edge.

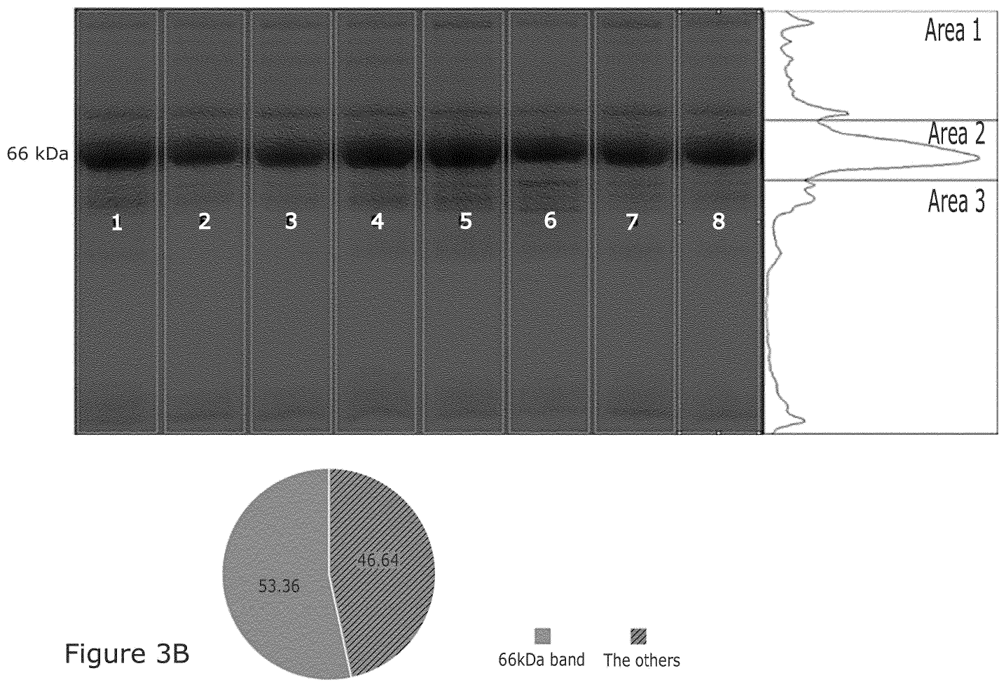

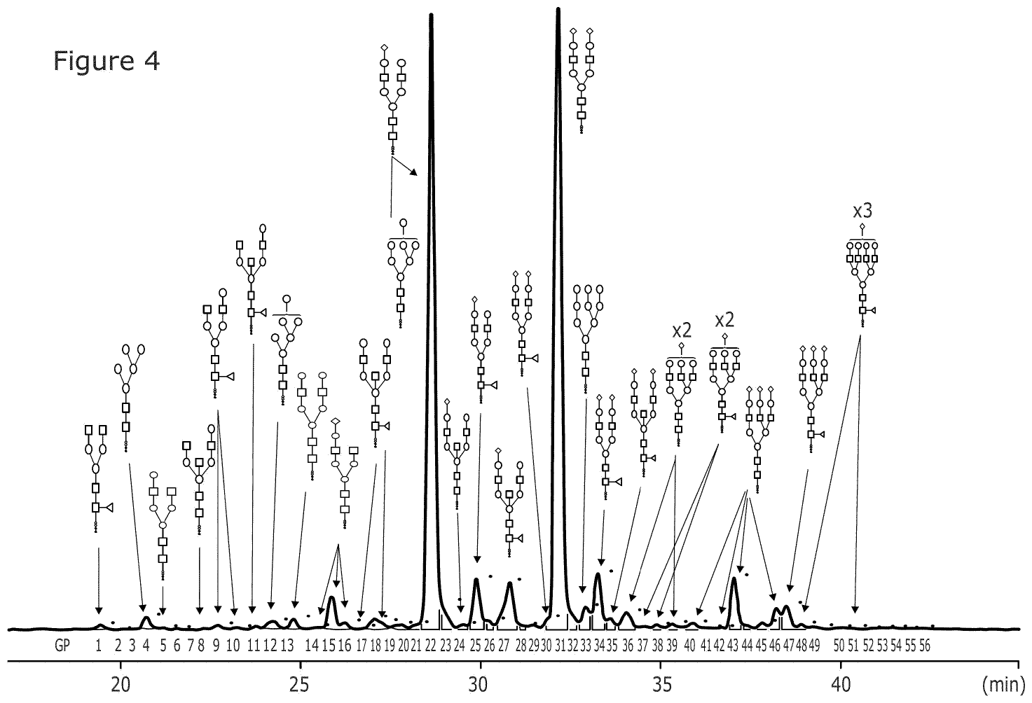

FIG. 4: Glycan profile of silica corona 3% plasma (unfinished). Released glycans from silica corona 3% plasma were labelled with procainamide for HILIC-FD-EIC-MS analysis. The chromatogram peaks were identified with HappyTools while the glycan structures were assigned with Bruker Compass DataAnalysis using MS1 and MS/MS. The glycan linkages of fucose and sialic acid are not specified. For a peak with multiple glycan structures detected, only the major structure is shown. The glycan structures were drawn with GlycoWorkbench and depicted following the Consortium for Functional Glycomics (CFG) notation: N-acetylglucosamine (blue square), fucose (red triangle), mannose (green circle), galactose (yellow circle) and N-acetylneuraminic acid (purple diamond).

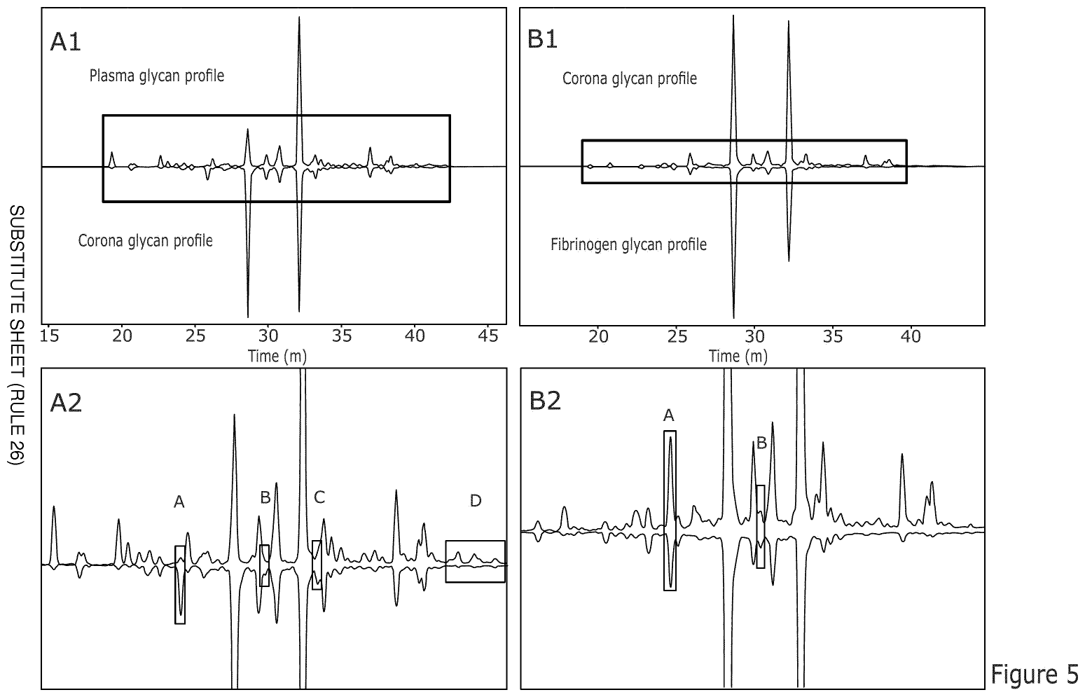

FIG. 5: Glycan profile of silica corona 3% plasma, in comparison with those of full plasma (A1-2) and fibrinogen (B1-2)—(unfinished). A1-2) The chromatograms of released glycans from plasma (up, blue) and silica corona 3% plasma (down, orange) were normalised to the highest peak intensity (GP31 in the corona's chromatogram). The zoomed-in area in the red box is shown in A2. Four areas enclosed by boxes show noticeable differences between the two glycan profiles, containing A2G1S1 (A); FA2G2S1 (B); M9 (C) and sialylated (fucosylated) tetra-antennary structures (D). B1-2) The chromatograms of released glycans from silica corona 3% plasma (up, orange) and fibrinogen (down, blue) were normalised to the highest peak intensity (GP22 in the corona's chromatogram). The zoomed-in area in the red box is shown in B2. The boxes show the shared glycans between the corona sample and fibrinogen control. The glycan structures are depicted as follows: N-acetylglucosamine (A), fucose (F), mannose (M), galactose (G) and N-acetylneuraminic acid (S).

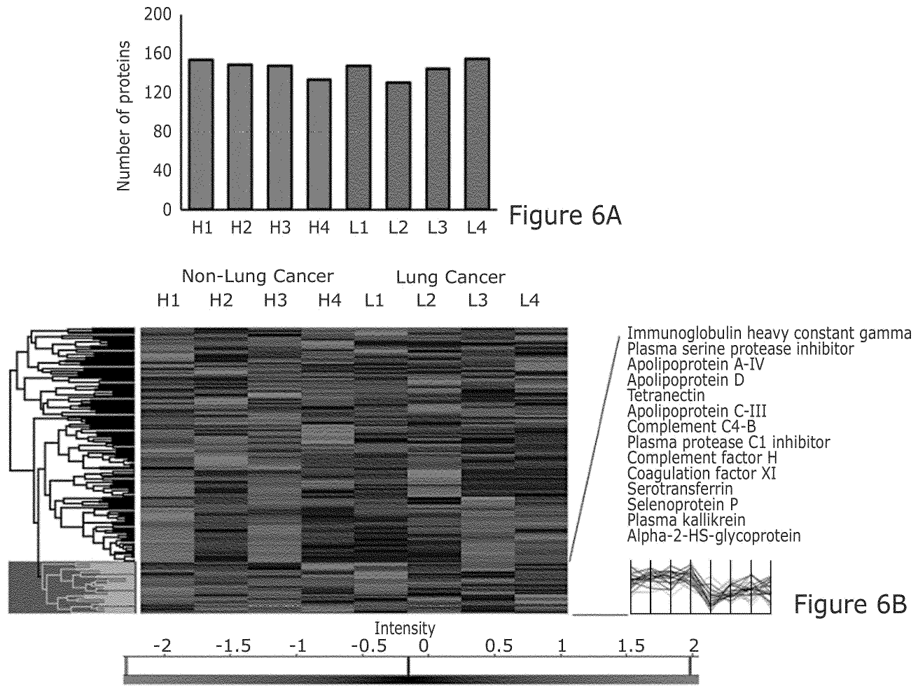

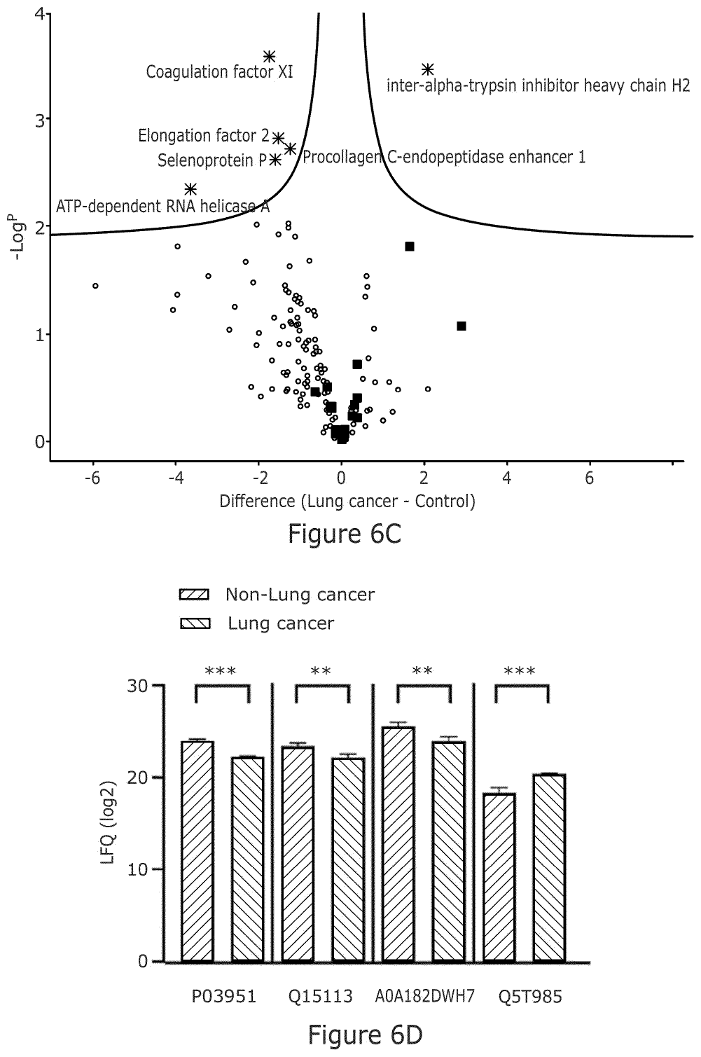

FIG. 6: Proteomic LFQ compares the lung cancer (L1, 2, 3, 4) and non-lung cancer samples (H1, 2, 3, 4). A) The numbers of proteins that met the LFQ normalization criteria (default in Maxquant). Missing values were later imputed from a normal distribution with the default width and down shift parameters. B) Protein heatmap of the samples after z-scoring. A protein cluster was expanded with some protein of interest and their intensity profiles (black). The analysis was performed in Perseus using Euclidean distance and default settings. C) Volcano plot with fold-change and p-values shows the t-test result between the two groups. The analysis was performed in Perseus, FDR=0.05, S0=0.1. Red: proteins more abundant in non-lung cancer group. Blue: proteins more abundant in lung cancer group. Star: 6 proteins with significant difference. Filled square: proteins in the top 20 abundances. D) LFQ intensity of the 4 secreted proteins with significant differences. Uniprot identifier: P03951: Coagulation factor XI, Q15113: Procollagen C-endopeptidase enhancer 1, A0A182DWH7: Selenoprotein P, Q5T985: Inter-alpha-trypsin inhibitor heavy chain H2. Error bars: Standard Error of the Mean (SEM). P values <0.01**; <0.001***.

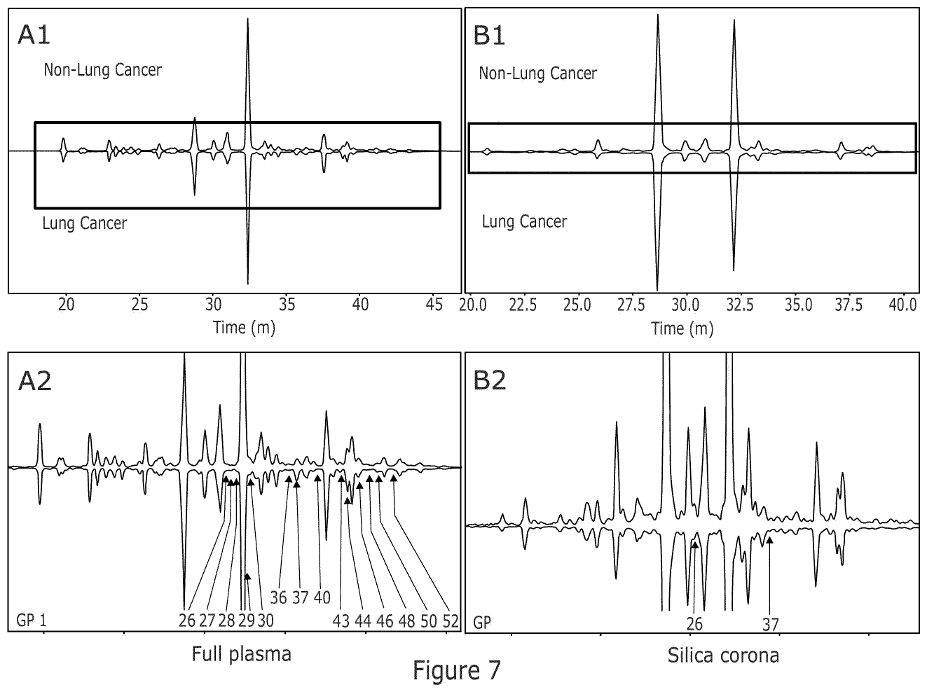

FIG. 7: Glycan profiles of full plasma (A1-2) and silica corona 3% plasma (B1-2), comparing the lung cancer and non-lung cancer groups. A1-2) The chromatograms of released glycans from full plasma of a non-lung cancer sample (up, orange) and lung cancer sample (down, blue) were normalised to the highest peak intensity (GP31 in the corona's chromatogram). The zoomed-in area in the red box is shown in A2. Glycan peaks, whose areas were significantly different between the two groups, are presented. Beside GP1, the other peaks can be grouped into sialylated biantennary (GP26-30) and sialylated (fucosylated) tri- and tetra-antennary structures (the remaining peaks). The filled colour of the numbers match that of the chromatogram, in which they are more abundant. B1-2) The chromatograms of released glycans from silica corona 3% plasma of a non-lung cancer sample (up, orange) and lung cancer sample (down, blue) were normalised to the highest peak intensity (GP22 in the corona's chromatogram). The zoomed-in area in the red box is shown in B2. Glycan peaks (GP), whose areas were significantly different between the two groups, are shown. The filled colour of the numbers match that of the chromatogram, in which they are more abundant.

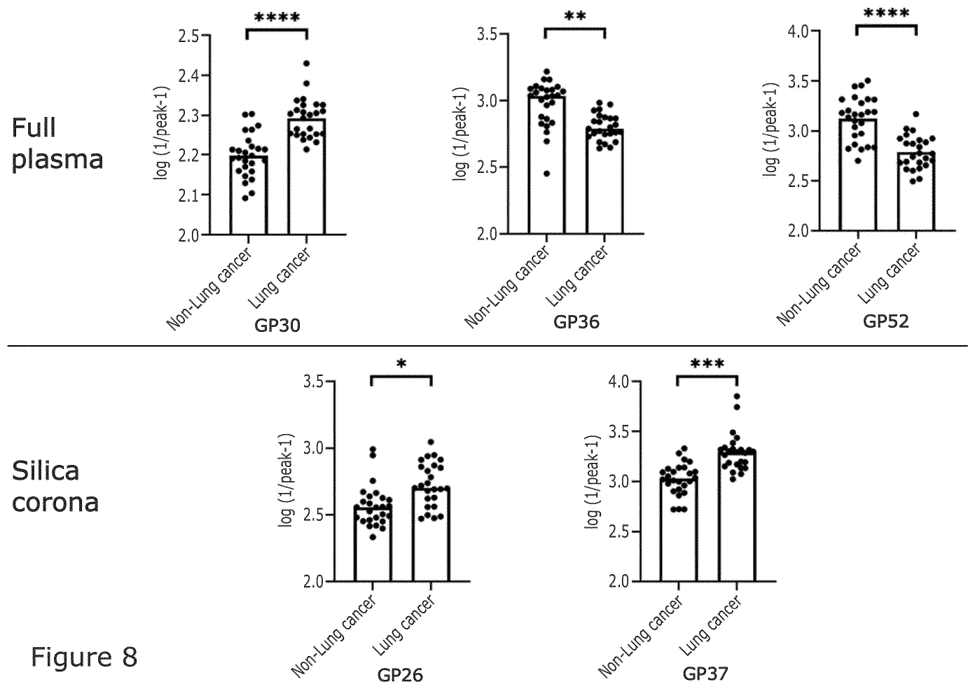

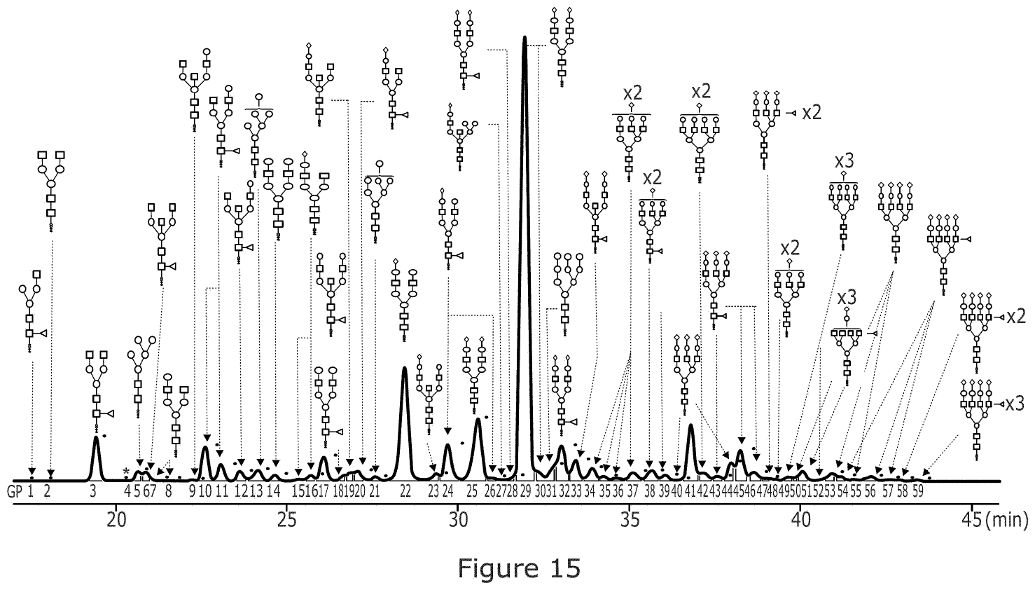

FIG. 8: Peaks with significantly different areas between the lung cancer and non-lung cancer groups. 3 glycan peaks (out of 14) were selected to show the differences between the full plasma glycan profiles of the two groups: A2BG2S2 (GP30), FA3G3S2 (GP36) and A4G4S3 (GP52). A representative chromatogram of full plasma samples with the peak annotation is in FIG. 15. There are only two glycan peaks from the silica corona that were found to be significantly different: FA2G2S1 (GP26) and FA3G3S2 (GP37). The normalised peak areas were log-transformed and checked for normality. Normally distributed data were compared by Student's t-test while non-normally distributed data were treated with Mann-Whitney U test. P-values were adjusted using Bonferroni correction. P values <0.05*; <0.01**, <0.001***; <0.0001****.

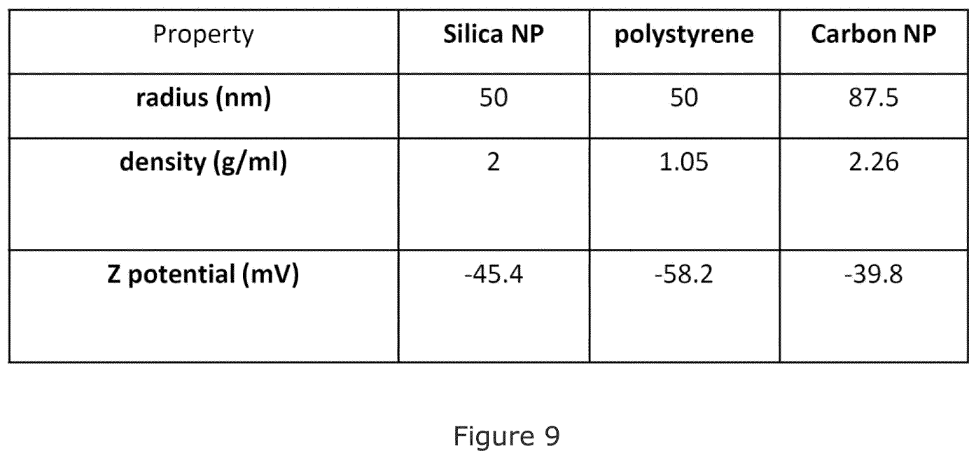

FIG. 9: Physicochemical properties of silica, polystyrene and carbon nanoparticles, including the particle radius (nm), density (g/ml) and Zeta potential (mV).

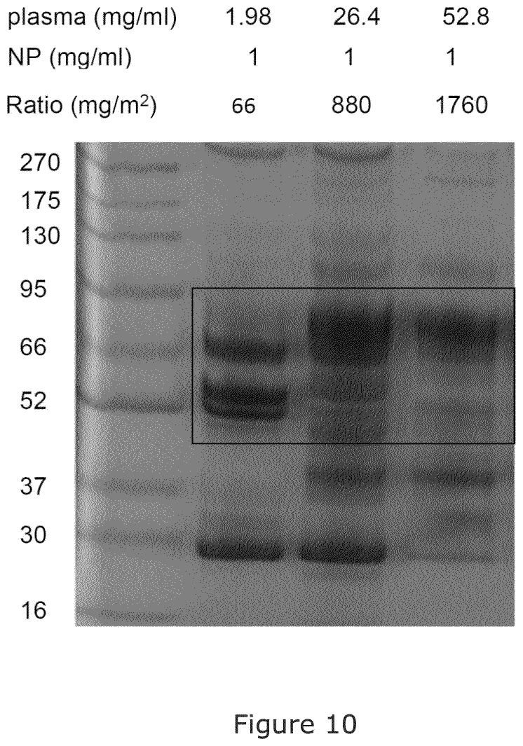

FIG. 10: 100 nm Kisker silica were incubated with human plasma. 3 data points showing the displacement of fibrinogen when increasing the ratio from 66 to 1760. Fibrinogen was present at the ratio of 66.

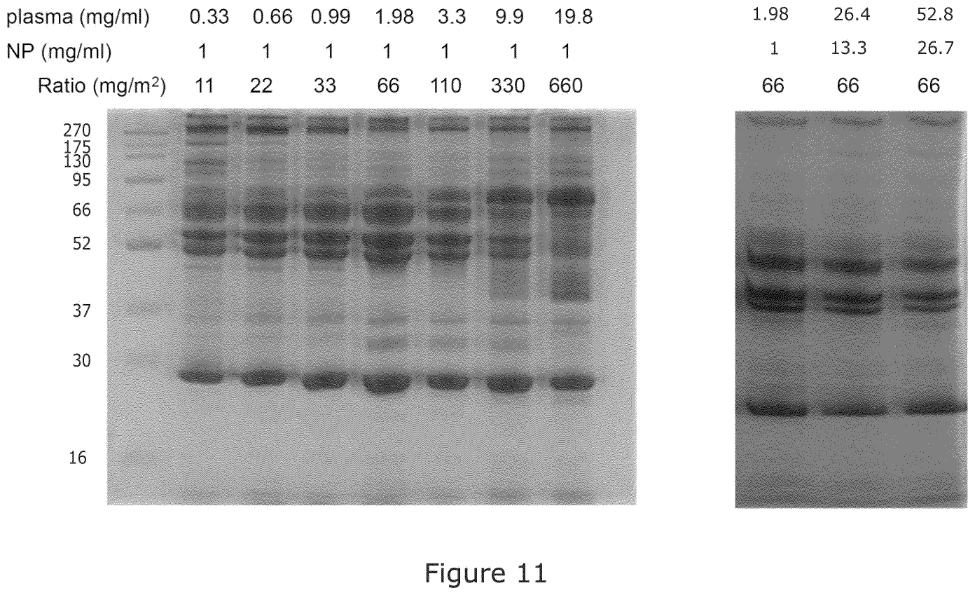

FIG. 11: 100 nm silica NPs were incubated with human plasma at more protein/NP concentration ratios Fibrinogen enrichment range: ratio between 11 and 330 mg/m² Histidine rich glycoprotein, kininogen-1 or plasminogen at ratio higher than 330 mg/m²

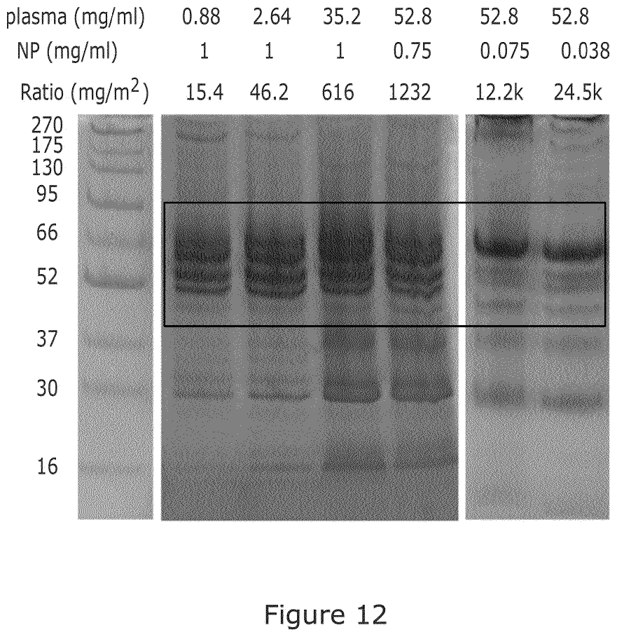

FIG. 12: 100 nm polystyrene NPs were incubated with human plasma at more protein/NP concentration ratios. Fibrinogen enrichment range: ratio below 12,200 mg/m²

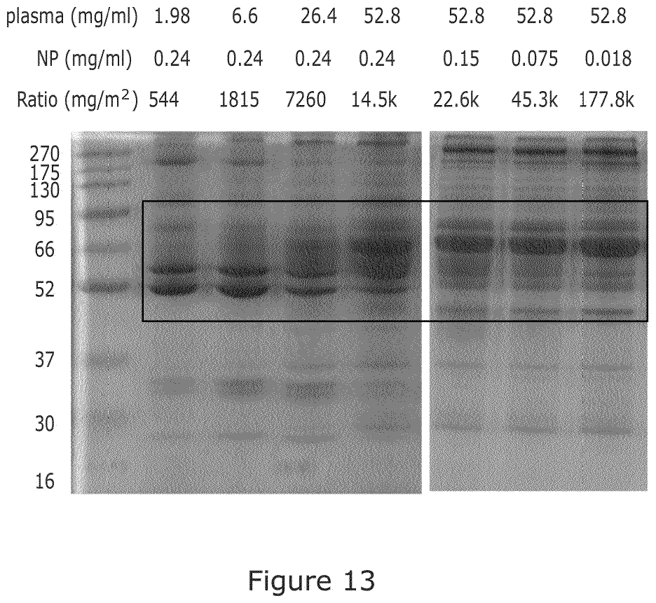

FIG. 13: 175 nm carbon NPs were incubated with human plasma at more protein/NP concentration ratios. Fibrinogen enrichment range: ratio below 22,600 mg/m²

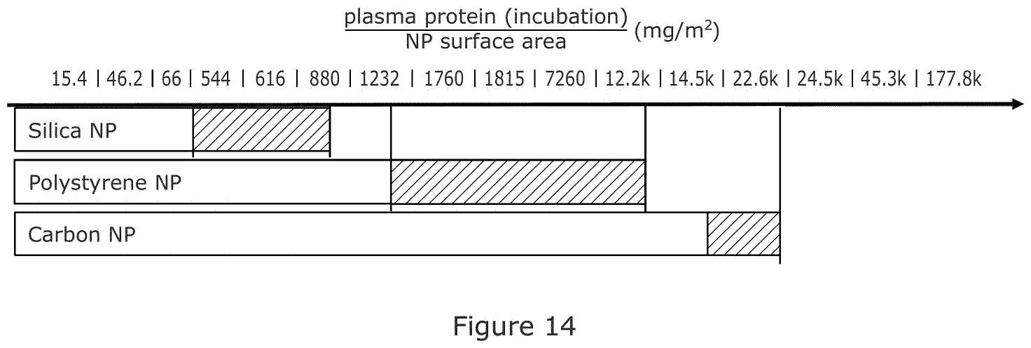

FIG. 14: The protein bands of fibrinogen chains in the SDS-PAGE gels of corona disappeared at high protein/NP surface ratio, depending on the type of NPs. The regions of protein/NP ratio where fibrinogen was no longer visible are shown for each of the three NPs: silica, polystyrene and carbon NPs (red).

FIG. 15: Released glycans were labelled with procainamide for HILIC-EIC-MS analysis. The glycan linkages of galactose, fucose and sialic acid are not specified. For a peak with multiple glycan structures detected, only the major structure is shown. The glycan structures were drawn with Glycoworkbench and depicted following the Consortium for Functional Glycomics (CFG) notation: N-acetylglucosamine (blue square), fucose (red triangle), mannose (green circle), galactose (yellow circle) and N-acetylneuraminic acid (purple diamond). *: GP4 contains a contaminant structure, whose structure is not shown in the figure.

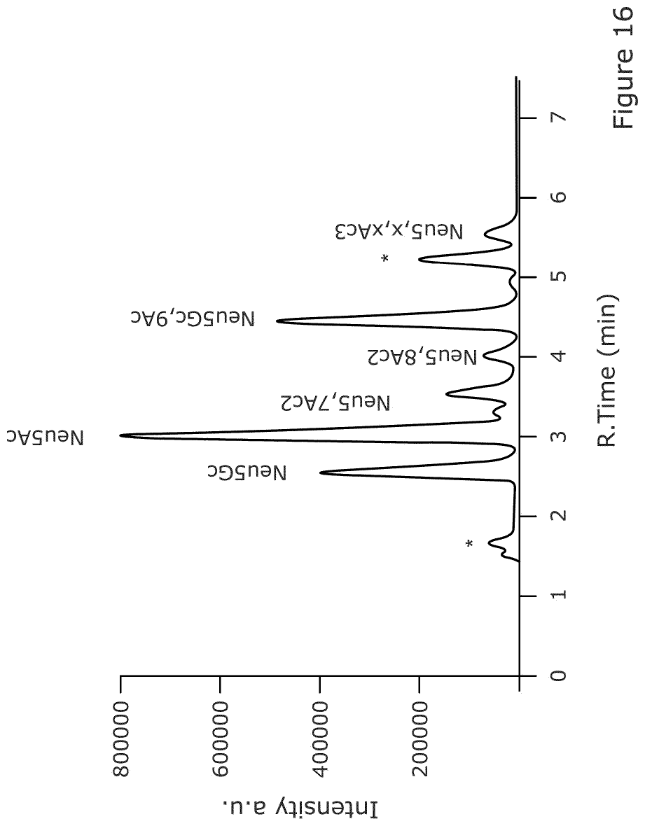

FIG. 16: Sialic Acid reference standard (SRP) run on an UHPLC-reverse phase chromatography.

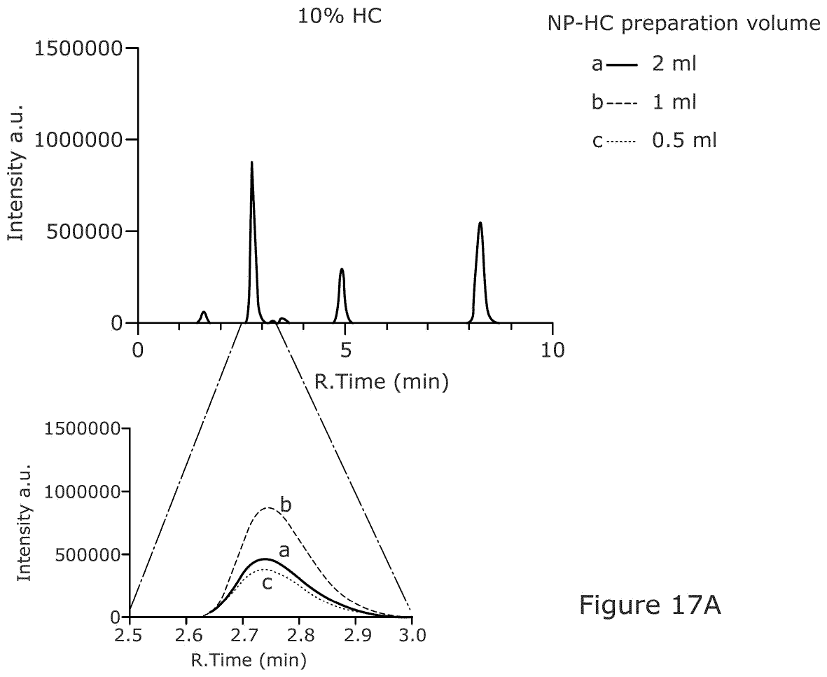

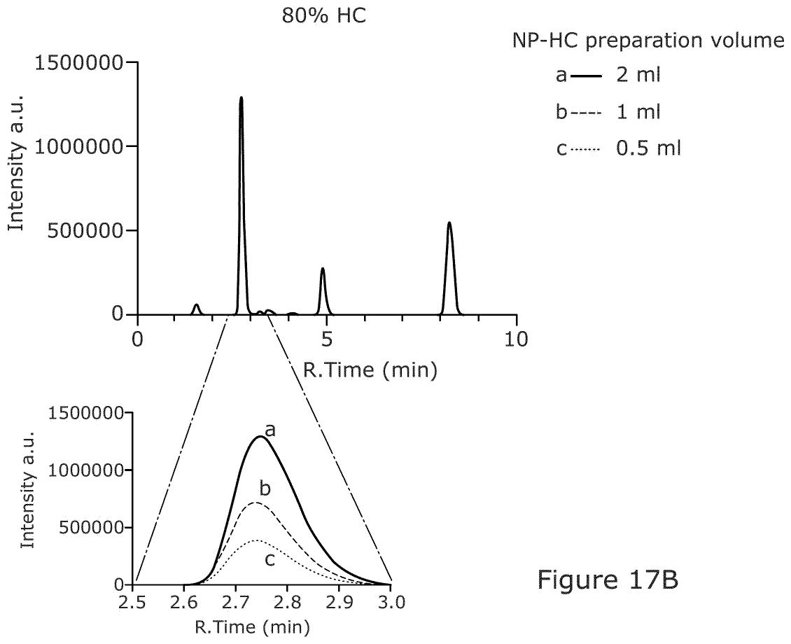

FIG. 17: sialic acid analysis from the NP-biomolecular corona at three different scaling up volume of 2 ml, 1 ml and 0.5 ml of 100 nm silica exposed to A) 10% and B) 80% of human plasma.

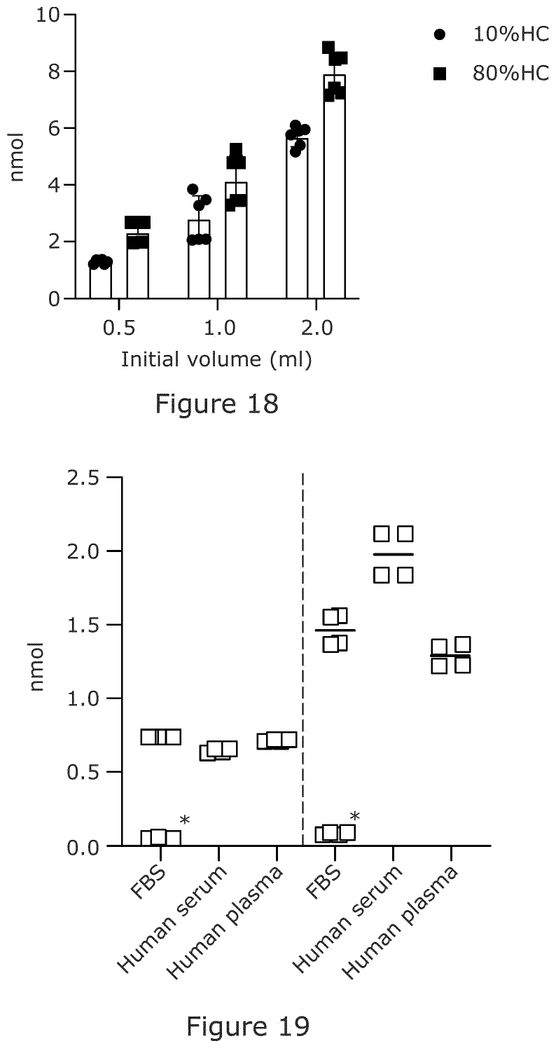

FIG. 18: Peak analysis of Neu5Ac after NP exposure to 10 and 80% of human plasma and at 3 different scaling up volumes.

FIG. 19: Sialic Acid analysis content analysis in the NP-corona samples after exposure to 10% (red labelled—left panel) and 80% (green labelled—right panel) corona after exposure to FBS, human serum and human plasma. Yellow box indicates Neu5Ac sialic acid (found in human) and the blue box (indicated by *) Neu5Gc sialic acid is found mostly in non-human biological media.

### EXAMPLES

### Example 1—Nanoparticle Protein Corona-Based Enrichment of Plasma Glycoproteins for N-Glycan Profiling and Application in Biomarker Discovery

### Summary

Biomolecular corona formation emerged as a recurring and important phenomenon in nanomedicine that has been investigated for potential applications in disease diagnosis. In this study, we made the first attempt to link the ‘personalised protein corona’ to the N-glycosylation profiling that has recently gained considerable interests in the biomarker discovery of human plasma as a powerful early warning biomolecules for chronic disease or for patient stratification. We visualized the protein corona formation could be exploited as an enrichment step that is critically important in both proteomic and proteoglycomic workflows. By using silica nanoparticles, plasma fibrinogen was enriched to a level in which its proteomic and glycomic ‘fingerprints’ could be traced with confidence. Although being simplified considerably compared to the glycan profile of full plasma, the corona glycan profile revealed new interesting information, particularly a fibrinogen-derived glycan peak of FA2G2S1 isomer that was found to potentially distinguish lung cancer patients in a pilot study.

### Results and Discussion

Plasma Protein Enrichment Method with Silica Nanoparticles.

100-nm silica NPs (1 mg/ml) were incubated with pooled plasma (3%, v/v) to form the silica corona. After one-hour incubation, the NP-corona complexes were separated from free plasma proteins and washed by centrifugation following with pellet resuspension in PBS. Different numbers of washes were used, up to three, as it has been shown that hard corona could be obtained after three centrifugal washes (20). Table 1A shows that the silica pristine NPs were colloidally stable with a Z-average of 114.2 nm and a PDI of 0.03; and an increase of hydrodynamic diameters was observed with the NP-corona complexes as expected while the colloidal stability was retained. All corona samples appeared as a single peak that was broader than that of the pristine NPs (FIGS. 1A and B).

TABLE 1
A) Hydrodynamic sizes of pristine silica NPs, silica corona
3% plasma after 1, 2 and 3 centrifugal washes, DLS measurements
(n = 3). SD: standard deviation. B) Hydrodynamic sizes
of the same samples, NTA measurements (n = 3). C) Apparent sizes
of the same samples, DCS measurements. In Table B and C, the sizes
of the tallest and 2^{nd} tallest peaks are shown.
A)

| | Z-Average, | Polydispersity |
|---|---|---|
| Sample | nm (±SD) | Index (±SD) |
| Silica nanoparticle | 114.2 ± 1.2 | 0.03 ± 0.02 |
| Silica corona, 1 wash | 175.1 ± 0.9 | 0.14 ± 0.02 |
| Silica corona, 2 washes | 174.3 ± 1.6 | 0.16 ± 0.01 |
| Silica corona, 3 washes | 169.8 ± 2.0 | 0.15 ± 0.02 |

| | | |
|---|---|---|
| | Main peak | Second peak |
| Sample | size, nm | size, nm |

B)

| | | |
|---|---|---|
| Silica nanoparticle | 111.0 | 161.0 |
| Silica corona, 1 wash | 131.0 | 183.0 |
| Silica corona, 2 washes | 127.0 | 171.0 |
| Silica corona, 3 washes | 125.0 | 179.0 |

C)

| | | |
|---|---|---|
| Silica nanoparticle | 99.3 | 116.4 |
| Silica corona, 1 wash | 95.1 | 111.8 |
| Silica corona, 2 washes | 95.1 | 111.5 |
| Silica corona, 3 washes | 94.5 | 110.9 |

To investigate further the size distributions of the samples, we used higher resolution techniques, including nanoparticle tracking analysis (NTA) and differential centrifugal sedimentation (DCS). The size distributions and peak sizes are shown in Table 1B, C and FIG. 1. The NTA shows the corona's main peak size of 131.0 nm, which is 20 nm bigger than the pristine NPs. The hydrodynamic size distributions of samples with different washes were quite similar to each other with a small decrease in the main peak size. Meanwhile, in the DCS, the major peak (95.1 nm) was shifted to the left in relation with the pristine silica (99.3 nm, FIG. 1C). Reducing plasma concentration below 3% or increasing NPs' concentration led to less stable coronas with decreasing percentage of this population (data not shown). Increasing number of washes did not affect significantly the particle size distribution, but slightly reduced the particle population with the size of 111.8 nm (FIG. 1D). A progressive decrease of the NP size occurred with the increasing numbers of washes, which indicates the loss of loosely bound proteins.

SDS-PAGE, however, shows a shared protein pattern for all the corona samples, with a band at about 30 kDa and triple bands between 45 and 70 kDa (FIG. 1E). The results indicate that there is a subtle difference between the coronas obtained after different centrifugal washes. Centrifugation is a simple and fast method to separate protein corona but it can remove loosely-bound proteins of the soft corona. Hence, we decided to keep as much information of plasma as possible with the one-wash 3% plasma corona protocol.

### Proteomic Features of Fibrinogen-Enriched Corona.

Shotgun proteomics was performed to investigate the protein corona composition of the silica corona at the plasma/NP surface of 66 mg/m². A total of 291 proteins was identified in the corona sample. FIG. 2A shows its 20 most abundant proteins, with ApoA1, fibrinogen and ApoB100 being at the top. The presence of these proteins can be observed on the SDS-PAGE gel at 28 kDa, triple bands between 45-70 kDa and high MW bands near 270 kDa, respectively (FIG. 2B). The most abundant plasma protein albumin is not in the top 10 of the corona protein composition. Densitometry was performed on SDS-PAGE gel images of 8 plasma samples from healthy individuals. FIG. 3A shows the corona's protein profile with 4 distinctive peaks containing fibrinogen and ApoA1. In total, fibrinogen could cover up to about 60% the protein signals. Meanwhile, in full plasma's protein profile, the single band at 66 kDa of albumin was dominant, accounting for nearly 50% of the total signals (FIG. 3B).

From the enrichment analysis, overall, the corona contains mainly proteins related to humoral immune response and coagulation processes, accounting for 34.5% and 24.6% of the GO terms respectively (FIGS. 2C and D). However, taking into account the top abundance list, the protein groups that likely represent the silica corona 3% plasma are lipoproteins (ApoA1, ApoB100, ApoE, ApoA4 and ApoC1) and cogulation related proteins (fibrinogen, histidine-rich glycoprotein, kininogen-1 and factor XII).

In terms of glycosylation, most of these top 20 proteins are N-glycosylated, particularly fibrinogen, ApoB100, histidine-rich glycoprotein and kininogen-1. Although being the most abundant protein in the corona, ApoA1 (28 kDa) is not N-glycosylated, but involved in glycation in specific disease conditions (21).

### Glycan Profile of Fibrinogen—Enriched Protein Corona.

To study the glycan profile of the silica corona, denatured corona glycoproteins were treated with PNGaseF, which cleaves the linkage between the core N-Acetylglucosamine (GlcNAc) and the asparagine residue on proteins, releasing all N-glycans except those containing fucose α1-3 linked to the reducing terminal GlcNAc (22). The glycans were labeled with procainamide before analysed by HILIC chromatography coupled with a fluorescence detector and electrospray ionisation MS (HILIC-FD-EIC-MS). In a 70-min HPLC gradient, 56 peaks were identified, whose glycan structures are shown in FIG. 4. For comparison, the glycan profile of full plasma containing 59 peaks is shown in Figure S2 (Supplementary Information). Noticeably, glycan peak A2G2S1 was highly present in the corona's glycan profile, which can be attributed to the enrichment of fibrinogen. Fibrinogen is a circulating glycoprotein synthesized by the liver hepatocytes. Among its three peptide chains, only β- and γ-chains are N-glycosylated, predominantly with A2G2S1 (53%) and A2G2S2 (33%) (23). In the silica corona, A2G2S1 peak accounted for 35.01% of the total N-glycome, a significant increase from only 11.42% in the full plasma profile (Figure S3, Supplementary Information).

Looking closer at the chromatograms of full plasma and silica corona, we identified 4 regions that are visually different between the two profiles (FIG. 5A1-2). In the corona sample, region A contains a strong signal of peak GP15 (A2G1S1); region B shows a peak splitting of a FA2G2S1 isomer; region C shows a peak splitting of M9; and region D contains diminished signals of sialylated (fucosylated) tri- and tetra-antennary structures, all in comparison with the full plasma. The chromatograms of the full plasma and corona samples were normalised by the highest peak intensity to facilitate the comparison between them. In each chromatogram, the relative abundance between the peaks was also preserved.

Beside A2G2S1 and A2G2S2, fibrinogen carries other minor glycan structures, including FA2G2S1 (4.38%), A2G2 (4.26%) and A2BG2S2 (1.01%) (23). By profiling the N-glycans of a fibrinogen control, we could confirm that the glycan peak in region B were contributed by this protein (FIG. 5B1-2) while that in region C was from other glycoproteins in the corona, most likely ApoB100 (24). The peak A2G1S1 in region A can also be seen in the fibrinogen control although this structure was not reported in literature before. Additionally, there were several unknown small peaks eluted before GP13 (A2G2), which indicates glycan contaminants could be present in the purchased fibrinogen (FIG. 5B1-2). As a result, we concluded that the glycan peaks in region A might be originated from other glycoproteins but not fibrinogen in the silica corona. The decrease in highly sialylated branched structures could be attributed to the absence in the corona of some plasma proteins carrying them, particularly alpha-1-acid glycoprotein.

### Fibrinogen Enrichment Method Applied in Biomarker Discovery of Lung Cancer.

Lung cancer is the principal cause of cancer-related death worldwide, causing up to three million deaths annually (25). It is a complex cancer with different subtypes and stages. Histologically, 80-85% of lung cancers are classified as non-small cell lung cancer (NSCLC), while the remaining is small cell lung cancer. The major subtypes of NSCLC are adenocarcinoma, squamous cell carcinoma and large cell carcinoma (26). As the survival rate of lung cancer patients increases significantly if early diagnosed (27), a non-invasive diagnostic procedure for this disease, particularly a plasma biomarker, is highly sought.

Plasma glycan changes, particularly increased fucosylation, highly sialylated and branched glycan levels have been linked to lung cancer, especially in the late stages (28-30). In this pilot study, 25 plasma samples of patients diagnosed with different types of lung cancer, mainly adenocarcinoma (15 units) and squamous cell carcinoma (7 units), were processed with silica NPs to enrich fibrinogen and compare with the non-lung cancer group. The sample information, including age, sex and total plasma protein concentrations determined by Bicinchoninic acid assay (BCA), is shown in Table 2. For each corona sample, the protein/NP concentration ratio was set at 1.98, which is equivalent to the condition used in the silica corona 3% plasma described above. Both full plasma and corona released glycans were analysed while quantitative MS was only used for the protein corona samples.

TABLE 2
Cohort sample information. Descriptive information for 26 non-lung
cancers and 25 lung cancers. The continuous variables Age and
Total protein concentration are shown as medians and interquartile
ranges while categorical feature Sex uses the basic counts.

| | Non-lung cancer | Lung cancer |
|---|---|---|
| Feature | (n = 26) | (n = 25) |
| Age (median [IQR]) | 63 (59-67.5) | 72 (66-73) |
| Sex (Male/Female) | 10/16 | 10/15 |
| Total plasma protein | 65.9 (63.4-73.48) | 79.62 (75.42-85.11) |
| concentration in mg/ml | | |
| (median [IQR]) | | |

Firstly, the corona sizes were characterised by DLS. The method was found to be highly compatible to the cohort plasma in terms of the colloidal stability as the majority of samples were stable with PdI below 0.25 (Table 3). There was no noticeable difference between the two groups. It is important to ensure the stability of the samples obtained with the biomolecular corona formation so that variations in the corona's protein composition and their relative amounts were better controlled.

TABLE 3
DLS size summary of the cohort. The colloidal stability of the samples
from the two groups are comparable with a minority of the samples
was categorised as unstable. Hydrodynamic size and Pdl are shown
as medians and interquartile ranges. A Pdl threshold of 0.25 was
used, below that the corona samples were considered stable.

| | Non-lung cancer | Lung cancer |
|---|---|---|
| Feature | (n = 26) | (n = 25) |
| Hydrodynamic size in nm | 194.1 (188.3-202.2) | 192.6 (185.3-210.5) |
| (Median [IQR]) | | |
| Pdl (Median [IQR]) | 0.15 (0.13-0.19) | 0.14 (0.11-0.18) |
| Number of samples | 25 | 23 |
| with Pdl < 0.25 | | |
| Number of samples | 1 | 2 |
| with 0.25 < Pdl < 0.35 | | |

After that, label free quantification (LFQ)-based MS strategy was performed in Maxquant to compare the protein abundance between the two groups (n=4). LFQ intensities are normalized median mass spectra intensity values that allow this quantification to be performed with any peptide and protein fractionation while maintaining high accuracy (31). There were between 130-155 proteins (out of 291) in each sample that could be used for the intensity-based comparison (FIG. 6A). The protein abundance patterns of the two groups shown in the heatmap were found to be quite heterogeneous but a protein cluster stood out as being enriched in the corona of the non-lung cancer group. This cluster was highlighted in pink colour with the names and intensity profiles of some protein members (FIG. 6B). They include different N-glycoproteins, for example Ig gamma-1 chain, ApoD, coagulation factor XI, transferrin, selenoprotein P and fetuin. Multiple t-test using False Discovery Rate (FDR) correction was performed between the two groups on Perseus. Volcano plot shows only one protein enriched in the lung cancer corona (inter-alpha-trypsin inhibitor heavy chain H2—ITIH2) while 5 proteins were more abundant in the controls (FIG. 6C). The LFQ intensity values of 4 significantly different secreted proteins; ITIH2, coagulation factor XI, procollagen C-endopeptidase enhancer 1 and selenoprotein P; were plotted in FIG. 6D. Among them, ITIH and selenoprotein P gene expressions in lung tumour tissues have been studied. ITIH is a protease-inhibiting glycoprotein that acted as an anti-inflammatory agent (32). The gene coding this protein was found to be downregulated in lung cancer tissue (33). On the other hand, selenoprotein P is a selenium-containing glycoprotein with at least 3 confirmed N-glycosites, playing important roles in selenium metabolism and antioxidative defence (34). Decrease in selenoprotein P causes dysfunctions related to oxidative stress and down-regulation of its gene SEPP1 in the lung tissue of NSCLC patients has also been reported (35).

Next, the glycan peak abundance in the chromatograms was compared by using HappyTools that integrated the peaks based on user-defined analyte lists. The peak areas were normalized by the total, then log-transformed before further analyses (4). Various physiological and behavioral parameters such as age, sex, body mass index, and several environmental factors, including smoking have been shown to associate with protein glycosylation (3). For both full plasma and corona glycan datasets, the association of peak areas with the age and gender were checked before further analyses and no relationship between them was established.

In the full plasma sample analysis, 15 glycan peaks were found to be significantly different between the two groups (Table S4, Supplementary Information). Most of them were more abundant in lung cancer samples, except GP29 and 30 (FIG. 7A1-2). Apart from GP1, these glycan peaks can be grouped into sialylated biantennary (GP26-30) and tri- or tetra-antennary (GP36-52) structures. It is known that branching is blocked by the insertion of a GlcNAc residue at a bisecting position between two arms by GlcNAc transferase III, resulting in no bisecting structures of tri- and tetra-antennary complexes (36). Therefore, the level of bisecting structures could vary in opposite direction to that of the branched glycans, which is seen with A2BG2S2 of peak GP30. Although a glycan derived trait analysis was not performed due to the coelution of multiple glycan structures, individual peak comparison demonstrates the upregulation of highly sialylated, branched glycans that have been previously reported in lung cancer plasma. However, some peaks containing traces of biantennary fucosylated isoforms (GP26 and GP28) were found to increase in lung cancer plasma, whose glycan trait was previously reported to decrease (29).

On the other hand, in the corona glycan profile analysis, only two peaks were found to be significantly different between the two groups, including FA2G2S1(GP26) and FA3G3S2 (GP37). Both of them were less abundant in the lung cancer samples (FIG. 7B1-2). The differences of some glycan peaks from both full plasma and corona analyses are shown in FIG. 8. After the log-transform, the larger the values were observed, the smaller the peak areas originally were. Interestingly, GP26 is the peak emerged in region B of the corona glycan profile (FIG. 5) that was not resolved in the full plasma profile. The selective glycoprotein enrichment by biomolecular corona formation diminished certain glycan structure abundance to reveal smaller adjacent ones, which could be the reason behind the emergence of GP26 and GP33 in the silica corona glycan profile. As GP26 was likely originated from the enriched fibrinogen (FIG. 5B), the finding indicates that FA2G2S1 glycoform of fibrinogen could be specifically altered in the lung cancer disease state. In the full plasma analysis, increased FA3G3S2 (GP36 and 40) in the lung cancer group was found. Meanwhile, the abundance of this glycoform (GP37) was decreased in the silica corona protein subset of lung cancer plasma. Although the connection between the overexpression of fucosyltransferases in cancer diseases and increased glycan fucosylation is straightforward and well-established, decreases in fucosylation were also reported in specific cancer types and glycan structures, which implies a complex interplay between multiple glycosyltransferases during the disease progress (4, 29, 37). Additionally, changes in glycosylation could also alter the stability and conformation of proteins, which likely affects their binding affinity to NPs. Beside FA3G3S2, we could not observe any changes in the highly sialylated, branched glycan group in the corona samples, which was found to be a noticeable lung cancer-related glycan alteration in full plasma.

In relation to the proteomic analysis (FIG. 6D), except procollagen C-endopeptidase enhancer 1, all of the proteins are reportedly N-glycosylated. For example, ITIH2 carries a biantennary glycan structure, with or without sialic acid moieties (38). Coagulation factor XI has 4 glycosites, containing mostly biantennary glycans [A2G2S2 (66.1%), A2G2S1 (19.8%), FA2G2S1 (0.9%)] with minor tri-antennary structures (5.4%) (39). However, due to their low abundance, it would be unlikely that their enrichment differences could be observed in the global glycan profile of the silica corona. More target-specific approaches on these proteins will be considered for future studies.

Several studies have isolated fibrinogen from human plasma to investigate its N-glycosylation in association with diseases, particularly by protein precipitation (40, 41). Although being simple and quick, the method depends on the dehydration of proteins, which likely results in the co-precipitation of fibrinogen with different proteins. The protein composition and reproducibility of the method were not specified in these studies. Immunoaffinity chromatography was also used to isolate fibrinogen from plasma in a semi high-throughput workflow with good reproducibility (42). The cost of monoclonal antibodies, however, would need to be considered in a cohort analysis. Association between coagulation and lung cancer, particularly elevated plasma fibrinogen, has been reported (43, 44). In this study, we exploited the protein corona formation to enrich fibrinogen from lung cancer plasma with silica NPs. The method is simple and a workflow with higher throughput can be established, for example, with silica-coated iron oxide NPs. The enrichment of fibrinogen can be seen in both proteomic and glycomic profiling. Although no significant differences in corona's fibrinogen levels were found, the glycan profiling revealed a glycan change of FA2G2S1 related to this protein. Most of the peaks that were significantly different between the two groups had very low abundances and appeared in crowded areas of the chromatograms. Their signals could be hidden by highly similar glycoforms due to coelution, as is the case with FA2G2S1 isoform in the full plasma glycan profile. The ‘gold standard’ for glycan profiling, HILIC-HPLC, separates glycans mainly based on their hydrophilicity and degree of branching. It has the capability to separate isomers, but not in a complete manner, especially with complex samples (45). Reducing the plasma complexity by exploiting biomolecular corona is a feasible option to obtain a higher resolution separation as in this study, we demonstrated that some specific glycan structures could be separated better in the silica corona glycan profile than in the full plasma one. Another option that the future work with biomolecular corona enrichment method could focus on is to combine the protein corona enrichment with a mixed-mode chromatography setup, for example HILIC-AEX (anion exchange chromatography) that separates glycans based on both their polarity and charges, mostly from the sialic acid residues (46).

### CONCLUSION

Protein corona composition is known to vary depending on the types of NPs and biological fluids. In this study, we exploited a specific plasma protein/silica NP concentration ratio to obtain a corona enriched mainly with apolipoproteins and coagulation-related proteins, particularly fibrinogen. The features of enriched fibrinogen were well observed in both the proteomic and glycan profiles of the corona. The enrichment method was applied to a small cohort of lung cancer plasma as a proof of concept. We identified some glycoproteins and N-glycan peaks, particularly of FA2G2S1 and FA3G3S2, which were able to separate the disease samples from the non-lung cancer group.

### Materials and Methods

### Materials:

Silica NPs (100 nm, stock concentration of 50 mg/ml) were purchased from Kisker Biotech GmbH. Phosphate buffer saline (PBS) tablets, Eppendorf LoBind microcentrifuge tubes and fibrinogen were purchased from Sigma Aldrich. One PBS tablet was dissolved in 200 ml of ultrapure water to obtain 10 mM PBS (pH 7.4 at 25° C.). Trypsin Gold was purchased from Promega. Blue loading buffer pack was purchased from Cell Signaling Technology. BCA kit and Imperial protein stain solution were purchased from Thermo Fisher Scientific (TFS). Prime-Step prestained protein ladder was purchased from BioLegend. Human plasma from 8 healthy donors provided by the Irish Blood Transfusion Service (IBTS) was mixed in equal proportions to obtain an average pooled plasma. 25 lung cancer plasma [lung adenocarcinoma (15), squamous cell carcinoma (7), small cell lung cancer (1), larger cell lung cancer (1) and lung mesothelioma (1)] and 8 non-cancer plasma samples were collected from St. Vincent's University Hospital. 18 plasma samples of healthy donors were purchased from BioIVT to form the non-lung cancer group with these above 8 non-cancer samples. Both pooled plasma and cohort plasma's total protein concentrations (mg/ml) were measured with BCA, following the manufacturer's instructions.

### Silica Corona Sample Preparation:

Protein corona samples were prepared by incubating silica NPs with specific plasma concentrations in LoBind tubes. Plasma aliquots were fully defrosted at room temperature, then centrifuged at 16,000 RCF for 3 minutes to remove any protein aggregations. Plasma solutions were diluted with PBS keeping the final total plasma protein/NP concentration constant and equal to 1.98. The final total volume was 2.0 ml and NPs' concentration was 1.0 mg/ml. NPs were allowed to incubate with the plasma solutions at 37° C. for one hour with continuous agitation. After the incubation in plasma, the samples were centrifuged for 10 minutes at 18,000 RCF, room temperature, to pellet the particle-protein complexes and separated from the supernatant plasma. The pellet was then resuspended in 500 μl of PBS and centrifuged again to pellet the biomolecular corona (1 wash). The procedure was repeated 1 and 2 times more to obtain 2 and 3—washed biomolecular coronas, respectively.

### Characterization of Silica Coronas:

DLS measurements at θ=173° were performed using a Zetasizer Nano ZS (Malvern). The sample cuvettes were equilibrated at 25° C. for 90 seconds. For each measurement, the number of run and duration were automatically determined and repeated three times. Data analysis has been performed according to standard procedures, and interpreted through a cumulant expansion of the field autocorrelation function to the second order.

NTA measurements were performed in static mode using a Nanosight NS300 (Malvern) equipped with 488 nm laser. Samples were diluted in PBS to a final volume of 1 ml, so that there were between 30-60 nanoparticles/frame. The camera (sCMOS) level was adjusted to have all particles distinctively visible while not saturate the detector. Each sample was recorded 3 times of 60 seconds each at 25° C. The sample was manually advanced between the recordings. The videos were analyzed by the in-built NanoSight Software NTA 3.2 using default settings.

Differential centrifugal sedimentation experiments were performed with a CPS Disc Centrifuge DC24000, using the standard sucrose gradient 8-24% (Analytik Ltd.). PVC calibration standard was used for each sample measurement. The time taken for spherical particles with homogenous density to travel from the centre of the disk to the detector can be directly related with the particle size. Meanwhile, if objects are inhomogeneous, or irregular in shape, the different arrival times still allow distinguish between the populations, although their sizes should only be considered as an ‘apparent’ size (47).

SDS-PAGE was performed as follows: immediately after the last centrifugation step, the protein corona pellet was resuspended in protein loading buffer following the manufacturer's instructions. The samples were boiled for 10 minutes at 100° C. and an equal protein amount was loaded in 12% polyacrylamide gel. Gel electrophoresis was performed at a constant voltage of 120 V, for about 60 minutes each, until the proteins neared the end of the gel. The gels were stained in the protein stain solution, following the manufacturer's guide. Gels were scanned using Amersham Imager 600 (GE Healthcare Life Sciences).

### Proteomic LC-MS/MS Sample Preparation and Analysis:

Eight MS samples were prepared as previously described (19). Non-lung cancer group included plasma samples from 3 healthy donors and 1 individual with negative lung cancer diagnosis. Cancer group consisted of 2 lung adenocarcinoma and 2 squamous cell cancer samples. All of them were selected randomly. LC-MS/MS was performed on a Dionex UltiMate3000 nanoRSLC coupled in-line with an Orbitrap Fusion Tribrid mass spectrometer (TFS). Briefly, the peptide samples were loaded onto the trapping column (PepMap100, C18, 300 μm×5 mm, 5 μm particle size, 100 Å pore size; TFS) for 3 minutes at a flow rate of 25 μL/min with 2% (v/v) acetonitrile, 0.1% (v/v) trifluoroacetic. Peptides were resolved on an analytical column (Acclaim PepMap 100, 75 μm×50 cm, 3 μm bead diameter column; TFS) using the following binary gradient; solvent A (0.1% (v/v) formic acid in LC-MS grade water) and solvent B (80% (v/v) acetonitrile, 0.08% (v/v) formic acid in LC-MS grade water) using 3-50% B for 45 minutes, 50-90% B for 5 minutes and holding at 90% B for 5 minutes at a flow rate of 300 nL/min before returning to 3% B. MS1 spectra were acquired over m/z 380-1500 in the Orbitrap (120 K resolution at 200 m/z), and automatic gain control (AGC) was set to accumulate 4×10⁵ ions with a maximum injection time of 50 ms. Data-dependent tandem MS analysis was performed using a top-speed approach (cycle time of 3 s), with precursor ions selected in the Quadrupole with an isolation width of 1.6 Da. The intensity threshold for fragmentation was set to 5000 and included charge states 2⁺ to 7⁺. Precursor ions were fragmented in the Orbitrap (30 K resolution at 200 m/z) using Higher energy Collision Dissociation (HCD) with a normalised collision energy of 28% and the MS2 spectra were acquired with a fixed first m/z of 110 in the ion trap. A dynamic exclusion of 50 s was applied with a mass tolerance of 10 ppm. AGC was set to 5×10⁴ with a maximum injection time set at 300 ms.

Protein identification and quantification were performed with Maxquant, version 1.6.17.0 (48). Using the Andromeda search engine, the MS/MS spectra were searched against the forward and reverse human Uniprot sequence database, accessed on Jun. 16, 2021 (https://www.uniprot.org). Cysteine carbamidomethylation was set as fixed modification while variable modifications included N-terminal acetylation and methionine oxidation. For both protein and peptide levels, the FDR thresholds were set to 0.01 and only peptides with an amino-acid length of seven or more were considered. The search filtrations were done using a standard target-decoy database approach. Other important search parameters included a value of 0.02 Da for MS/MS mass tolerance, a value of 10 ppm for peptide mass tolerance and tolerance for the occurrence of up to two missed cleavages. The LFQ was restricted to proteins identified with at least two unique peptides. Additionally, for a protein to be considered valid, two peptide ratios were needed.

Bioinformatic analysis was performed with Perseus software, version 1.6.5.0 (49). For the pooled silica corona dataset, log₂ intensities were used to rank proteins, while log₂ LFQ intensities were used for the cohort protein corona comparison. Imputation of missing values was done by random selection using a normal distribution with negative shift of 1.8 standard deviations from the mean and with a width of 0.3 standard deviations. These log₂ LFQ intensities values for all proteins were then used for heatmap presentations (after z-scoring) and statistical analysis. Proteome comparisons of the cohort coronas were done with t-test and FDR-corrected p-values were used for filtering significant abundance differences. The volcano plot was generated using the default settings (FDR=0.05, S0=0.1). The list of proteins identified in the silica corona 3% pooled plasma was exported to ClueGO/Cytoscape for gene ontology enrichment against Homo sapiens organism database (50). The ontology Biological Process was selected for the enrichment analysis and the corrected p-values were set to maximal 10⁻⁶ for the terms to be shown in the DAG.

### Sample Glycan Profiling:

Glycan release: the N-glycans were released from the protein corona using PNGaseF kit (Ludger Ltd.). Briefly, the corona was resuspended in 15 μL of ultrapure water. 10 μL of 10× denaturation solution was added to each sample and mixed. The samples were incubated for 10 minutes at 100° C. The sample tube was briefly vortexed and centrifuged at 18,000 RCF for 10 minutes to remove NPs. 20 μL of 10× reaction buffer, 20 μL of 10% NP-40 solution, 135 μL of pure water and 1 μL of PNGaseF were added to each supernatant containing glycoproteins. Samples were vortexed and incubated overnight at 37° C. (14-16 hours).

Fluorescent labelling: 200 μL of each sample was transferred to a non-skirted 96 well PCR plate (300 μL) and the samples dried down over 9 hours. The released N-glycans were converted to aldoses with 40 μL of 0.1% formic acid over 45 minutes, filtered through a 96-well protein binding plate and dried down completely over 9 hours. Released N-glycans were fluorescently labelled by reductive amination with procainamide using LudgerTag Procainamide Glycan Labelling Kit (Ludger Ltd.). Briefly, samples were incubated for 60 minutes at 65° C. with 20 μL of procainamide labelling solution. Purification of procainamide labelled glycans: The procainamide labelled N glycans were cleaned up using a HILIC-type purification Ludger-Clean Procainamide Clean-up Plate (Ludger Ltd.). The purified procainamide labelled N-glycans were eluted with pure water (300 μL).

LC-ESI-MS and MS/MS analysis: procainamide labelled samples and system suitability standards were analysed by HILIC-(U)HPLC-ESI-MS with fluorescence detection. To 25 μL of each sample was added 75 μL of acetonitrile. 25 μL of each sample was injected onto an ACQUITY UPLC BEH-Glycan 1.7 μm, 2.1×150 mm column (Waters) at 40° C. on an Ultimate 3000 UHPLC instrument with a fluorescence detector (λₑₓ=310 nm, λₑₘ=370 nm), attached to a Bruker Amazon Speed electron-transfer dissociation (ETD) instrument. The chromatography conditions used were: Solvent A was 50 mM ammonium formate pH 4.4 made from Ludger Stock Buffer, and solvent B was acetonitrile. Gradient conditions were: 0 to 10 min, 76 to 76% B at a flow rate of 0.4 mL/min; 10 to 85 min, 76 to 51% B at a flow rate of 0.4 mL/min; 85 to 89 min, 51 to 10% at a flow rate of 0.2 mL/min; 89 to 93 min, 10 to 76% at a flow rate of 0.2 mL/min; 93 to 95 min, 76 to 76% at a flow rate of 0.4 mL/min. The Amazon Speed settings were: source temperature 250° C., gas flow 10 L/min; Capillary voltage 4500 V; ICC target 200,000; max accu time 50.00 ms; rolling average 2; number of precursor ions selected 3, release after 1.0 min; Positive ion mode; Scan mode: enhanced resolution; mass range scanned, 300-1700; Target mass, 657.28.

Glycan structures were assigned with Bruker Compass DataAnalysis and GlycoWorkbench 2 software (51). The glycan structure compositions were identified by using the registered parent m/z values from the full MS scan. Potential glycan structures were then in-silico defragmented to generate their theoretical ion m/z. The calculated and registered m/z values from the MS/MS scan were then compared to confirm the presence of the structures. Peak integration was performed with HappyTools that did the peak calibration and integration by examining user-defined calibrant and analyte peak lists, respectively (52). For the calibration, we used 4-5 glycan peaks with high signal to noise ratios that were spaced out roughly equally in the chromatograms. The analyses were performed in two separate batches, one for all the full plasma chromatograms and the other for all protein corona chromatograms. Relative abundances of the peaks were obtained directly from the software outputs.

### Statistics and Data Plotting

Statistical analysis was performed in R Studio v1.1.463 (the R Foundation for Statistical Computing) running R version 4.0.4. Relative peak areas under the curve were log-transformed [log(1/peak−1)] and the normality of distribution was determined using the Shapiro-Wilk test. Normally and non-normally distributed data were compared using Student's T-test and Mann-Whitney's U-tests, respectively. Associations between sex and each log-transformed peak area were compared in univariate pairwise analyses. Associations between age and individual log-transformed peak areas were examined visually by scatter plot in the first instance, and then with generalised linear models incorporating sex and age as co-variates. To correct for multiple testing, p-values in the pairwise analyses were corrected using the Bonferroni method and were considered significant if <0.05.

Other data were analysed and plotted with ImageJ version 1.53c (Fiji package version 2.1.0), GraphPad Prism (version 9) and Excel (Office 2016). The abstract figure was made with ChemDraw (version 16.0).

### REFERENCES

1. Helenius A, Aebi M. Intracellular functions of N-linked glycans. Science. 2001; 291(5512):2364-9.
2. Stowell S R, Ju T, Cummings R D. Protein glycosylation in cancer. Annual Review of Pathology: Mechanisms of Disease. 2015; 10:473-510.
3. Dotz V, Wuhrer M. N-glycome signatures in human plasma: associations with physiology and major diseases. FEBS letters. 2019; 593(21):2966-76.
4. Doherty M, Theodoratou E, Walsh I, Adamczyk B, Stöckmann H, Agakov F, et al. Plasma N-glycans in colorectal cancer risk. Scientific reports. 2018; 8(1):1-12.
5. Vreeker G C, Wuhrer M. Reversed-phase separation methods for glycan analysis. Analytical and bioanalytical chemistry. 2017; 409(2):359-78.
6. Monopoli M P, Åberg C, Salvati A, Dawson K A. Biomolecular coronas provide the biological identity of nanosized materials. Nature nanotechnology. 2012; 7(12):779-86.
7. Xu M, Soliman M G, Sun X, Pelaz B, Feliu N, Parak W J, et al. How entanglement of different physicochemical properties complicates the prediction of in vitro and in vivo interactions of gold nanoparticles. ACS nano. 2018; 12(10):10104-13.
8. Hajipour M J, Laurent S, Aghaie A, Rezaee F, Mahmoudi M. Personalized protein coronas: a “key” factor at the nanobiointerface. Biomaterials Science. 2014; 2(9):1210-21.
9. Hadjidemetriou M, Papafilippou L, Unwin R D, Rogan J, Clamp A, Kostarelos K. Nano-scavengers for blood biomarker discovery in ovarian carcinoma. Nano Today. 2020; 34:100901.
10. Caracciolo G, Safavi-Sohi R, Malekzadeh R, Poustchi H, Vasighi M, Chiozzi R Z, et al. Disease-specific protein corona sensor arrays may have disease detection capacity. Nanoscale Horizons. 2019; 4(5):1063-76.
11. Ponomarenko E A, Poverennaya E V, Ilgisonis E V, Pyatnitskiy M A, Kopylov A T, Zgoda V G, et al. The size of the human proteome: the width and depth. International journal of analytical chemistry. 2016; 2016.
12. Anderson N L, Anderson N G. The human plasma proteome: history, character, and diagnostic prospects. Molecular & cellular proteomics. 2002; 1(11):845-67.
13. Wu C, Duan J, Liu T, Smith R D, Qian W-J. Contributions of immunoaffinity chromatography to deep proteome profiling of human biofluids. Journal of Chromatography B. 2016; 1021:57-68.
14. Timp W, Timp G. Beyond mass spectrometry, the next step in proteomics. Science Advances. 2020; 6(2):eaax8978.
15. Zaia J. Mass spectrometry and the emerging field of glycomics. Chemistry & biology. 2008; 15(9):881-92.
16. Cao Z, Tang H-Y, Wang H, Liu Q, Speicher D W. Systematic comparison of fractionation methods for in-depth analysis of plasma proteomes. Journal of proteome research. 2012; 11(6):3090-100.
17. Riley N M, Bertozzi C R, Pitteri S J. A pragmatic guide to enrichment strategies for mass spectrometry-based glycoproteomics. Molecular & Cellular Proteomics. 2021:100029.
18. Kailemia M J, Park D, Lebrilla C B. Glycans and glycoproteins as specific biomarkers for cancer. Analytical and bioanalytical chemistry. 2017; 409(2):395-410.
19. Soddu L, Trinh D N, Dunne E, Kenny D, Bernardini G, Kokalari I, et al. Identification of physicochemical properties that modulate nanoparticle aggregation in blood. Beilstein journal of nanotechnology. 2020; 11(1):550-67.
20. Monopoli M P, Pitek A S, Lynch I, Dawson K A. Formation and characterization of the nanoparticle-protein corona. Nanomaterial interfaces in biology: Springer; 2013. p. 137-55.
21. Calvo C, Ulloa N, Campos M, Verdugo C, Ayrault-Jarrier M. The preferential site of non-enzymatic glycation of human apolipoprotein Al in vivo. Clinica chimica acta. 1993; 217(2):193-8.
22. Freeze H H, Kranz C. Endoglycosidase and glycoamidase release of N-linked glycans. Current protocols in molecular biology. 2010; 89(1):17.3 A. 1-3 A. 25.
23. Adamczyk B, Struwe W B, Ercan A, Nigrovic P A, Rudd P M. Characterization of fibrinogen glycosylation and its importance for serum/plasma N-glycome analysis. Journal of proteome research. 2013; 12(1):444-54.
24. Clerc F, Reiding K R, Jansen B C, Kammeijer G S, Bondt A, Wuhrer M. Human plasma protein N-glycosylation. Glycoconjugate journal. 2016; 33(3):309-43.
25. Cho W C-S. Potentially useful biomarkers for the diagnosis, treatment and prognosis of lung cancer. Biomedicine & pharmacotherapy. 2007; 61(9):515-9.
26. Mukherjee A, Paul M, Mukherjee S. Recent progress in the theranostics application of nanomedicine in lung cancer. Cancers. 2019; 11(5):597.
27. Hoffman P C MA, Vokes E E. Lung cancer. Lancet. 2000; 355(9202):479-85.
28. Arnold J N, Saldova R, Hamid U M A, Rudd P M. Evaluation of the serum N-linked glycome for the diagnosis of cancer and chronic inflammation. Proteomics. 2008; 8(16):3284-93.
29. Arnold J N, Saldova R, Galligan M C, Murphy T B, Mimura-Kimura Y, Telford J E, et al. Novel glycan biomarkers for the detection of lung cancer. Journal of proteome research. 2011; 10(4):1755-64.
30. Adamczyk B, Tharmalingam T, Rudd P M. Glycans as cancer biomarkers. Biochimica et Biophysica Acta (BBA)-General Subjects. 2012; 1820(9):1347-53.
31. Cox J, Hein M Y, Luber C A, Paron I, Nagaraj N, Mann M. Accurate proteome-wide label-free quantification by delayed normalization and maximal peptide ratio extraction, termed MaxLFQ. Molecular & cellular proteomics. 2014; 13(9):2513-26.
32. Fries E, Kaczmarczyk A. Inter-alpha-inhibitor, hyaluronan and inflammation. Acta Biochimica Polonica. 2003; 50(3):735-42.
33. Hamm A, Veeck J, Bektas N, Wild P J, Hartmann A, Heindrichs U, et al. Frequent expression loss of Inter-alpha-trypsin inhibitor heavy chain (ITIH) genes in multiple human solid tumors: a systematic expression analysis. BMC cancer. 2008; 8(1):1-15.
34. Saito Y. Selenium Transport Mechanism via Selenoprotein P—Its Physiological Role and Related Diseases. Frontiers in Nutrition. 2021; 8:278.
35. Gresner P, Gromadzinska J, Jablonska E, Kaczmarski J, Wasowicz W. Expression of selenoprotein-coding genes SEPP1, SEP15 and hGPX1 in non-small cell lung cancer. Lung Cancer. 2009; 65(1):34-40.
36. Harpaz N, Schachter H. Control of glycoprotein synthesis. Processing of asparagine-linked oligosaccharides by one or more rat liver Golgi alpha-D-mannosidases dependent on the prior action of UDP-N-acetylglucosamine: alpha-D-mannoside beta 2-N-acetylglucosaminyltransferase I. The Journal of biological chemistry. 1980; 255(10):4894-902.
37. Saldova R, Asadi Shehni A, Haakensen V D, Steinfeld I, Hilliard M, Kifer I, et al. Association of N-glycosylation with breast carcinoma and systemic features using high-resolution quantitative UPLC. Journal of proteome research. 2014; 13(5):2314-27.
38. Flahaut C, Capon C, Balduyck M, Ricart G, Sautiere P, Mizon J. Glycosylation pattern of human inter-α-inhibitor heavy chains. Biochemical Journal. 1998; 333(3):749-56.
39. Faid V, Denguir N, Chapuis V, Bihoreau N, Chevreux G. Site-specific N-glycosylation analysis of human factor XI: Identification of a noncanonical NXC glycosite. Proteomics. 2014; 14(21-22):2460-70.
40. Nagel T, Klaus F, Ibanez I G, Wege H, Lohse A, Meyer B. Fast and facile analysis of glycosylation and phosphorylation of fibrinogen from human plasma-correlation with liver cancer and liver cirrhosis. Analytical and bioanalytical chemistry. 2018; 410(30):7965-77.
41. Baralić M, Gligorijević N, Brković V, Katrlík J, Pažitná L, Šunderić M, et al. Fibrinogen Fucosylation as a Prognostic Marker of End-Stage Renal Disease in Patients on Peritoneal Dialysis. Biomolecules. 2020; 10(8):1165.
42. Vidic U, Trbojević-Akmaičić I, Černigoj U, Albers M, Gašperšič J, Pučić-Baković M, et al. Semi-high-throughput isolation and N-glycan analysis of human fibrinogen using monolithic supports bearing monoclonal anti-human fibrinogen antibodies. Electrophoresis. 2017; 38(22-23):2922-30.
43. Jones J M, McGonigle N C, McAnespie M, Cran G W, Graham A N. Plasma fibrinogen and serum C-reactive protein are associated with non-small cell lung cancer. Lung cancer. 2006; 53(1):97-101.
44. Sheng L, Luo M, Sun X, Lin N, Mao W, Su D. Serum fibrinogen is an independent prognostic factor in operable nonsmall cell lung cancer. International journal of cancer. 2013; 133(11):2720-5.
45. Huang Y, Nie Y, Boyes B, Orlando R. Resolving isomeric glycopeptide glycoforms with hydrophilic interaction chromatography (HILIC). Journal of biomolecular techniques: JBT. 2016; 27(3):98.
46. Largy E, Cantais F, Van Vyncht G, Beck A, Delobel A. Orthogonal liquid chromatography-mass spectrometry methods for the comprehensive characterization of therapeutic glycoproteins, from released glycans to intact protein level. Journal of Chromatography A. 2017; 1498:128-46.
47. Pitek A S, O'Connell D, Mahon E, Monopoli M P, Baldelli Bombelli F, Dawson K A. Transferrin coated nanoparticles: study of the bionano interface in human plasma. PloS one. 2012; 7(7):e40685.
48. Tyanova S, Temu T, Cox J. The MaxQuant computational platform for mass spectrometry-based shotgun proteomics. Nature protocols. 2016; 11(12):2301-19.
49. Tyanova S, Temu T, Sinitcyn P, Carlson A, Hein M Y, Geiger T, et al. The Perseus computational platform for comprehensive analysis of (prote) omics data. Nature methods. 2016; 13(9):731-40.
50. Bindea G, Mlecnik B, Hackl H, Charoentong P, Tosolini M, Kirilovsky A, et al. ClueGO: a Cytoscape plug-in to decipher functionally grouped gene ontology and pathway annotation networks. Bioinformatics. 2009; 25(8):1091-3.
51. Ceroni A, Maass K, Geyer H, Geyer R, Dell A, Haslam S M. GlycoWorkbench: a tool for the computer-assisted annotation of mass spectra of glycans. Journal of proteome research. 2008; 7(4):1650-9.
52. Jansen B C, Hafkenscheid L, Bondt A, Gardner R A, Hendel J L, Wuhrer M, et al. HappyTools: A software for high-throughput HPLC data processing and quantitation. PloS one. 2018; 13(7):e0200280.

### Example 2

After been assessed the protein and glycan changes at the different surface area/protein amount ratio, we have further evaluated whether the glycan composition of the corona would differ in different exposing condition. For this purpose, we carried out a sialic acid quantification assay using a quantitative sialic acid analysis (Ludger). For this purpose, 100 nm silica nanoparticles were exposed to a different surface area/protein amount ratio using human plasma and foetal bovine serum (FBS), and the NP strongly bound corona complexes were isolated using centrifugation following washes to remove the loosely bound proteins. The sialic acid analysis was carried out following the manufacturer's protocol, where the monosaccharide was released by exposing the complex to acid media, followed by labelling with a fluorophore.

The labelled monosaccharides were then run on UHPLC chromatography by reverse phase chromatography. A sialic acid reference standard (SRP) was also run that contained known sialic acid types as they would elute at a different time from the column (FIG. 16).

To evaluate the sensitivity of the assay, 100 nm silica NP was exposed to different surface area/protein amount ratios, such as 0.5 mg/ml of NP to 10 and 80% of human plasma, and at 3 different scale up volumes, such as 0.5 ml, 1 ml and 2 ml to evaluate the assay sensitivity (FIG. 17). In both exposing conditions, a main peak at 3 ml was eluted by chromatography, which corresponds to N-Acetylneuraminic acid (Neu5Ac) which is the main sialic acid found in humans (FIGS. 17A and 17B). as expected, the peak intensity changes at each scale up volumes, indicating that the sialic acid content increased as we scaled up the experimental condition (as expected) but without reaching the plateau indicating a broad dynamic range of detection.

Additionally, the peak analysis also revealed that each NP-corona condition, contained a different amount of sialic acid, indicating that a different type and amount of glycoprotein are formed in each condition.

Sialic acid amount and type also varied significantly when the NPs were exposed to different biological fluids and types. In particular, Neu5Ac quantity was significantly lower at 10% compared to 80% corona (FIG. 19). These changes in sialic acid indicate that different glycoprotein species are isolated in each experimental condition. Variances in the sialic acid quantity also occurred when the NP were exposed to the same amount of proteins but different media, suggesting that a different prefractionation also occurred when changing the media during the incubation step. In addition, the presence of Neu5Gc was also detected in NP corona when exposed to FBS, while no Neu5Gc was detected in the unprocessed FBS, indicating that the corona led to the enrichment of glycoproteins carrying this glycan type.

1. A method for selective enrichment of glycoproteins from a sample comprising proteins, the method comprising:
determining the concentration of proteins in the sample or providing the sample with a defined concentration of proteins;
incubating particles in the sample to form a protein corona comprising glycoproteins bound to the surface of the particles, wherein the protein concentration to the total surface area of the particles is selected, and/or the particle material is selected, in order to enrich for a specific glycoprotein species on the protein corona; and
optionally isolating the protein corona from the sample.

2. The method according to claim 1, wherein the sample protein concentration to total surface area of the particles is 11 to 330 mg/m² or 660 to 1760 mg/m².

3. The method according to claim 1 or 2, wherein the method further comprises the step of determining the glycoprotein and/or glycan profile of the protein corona isolated from the sample.

4. The method according to any preceding claim, wherein the particles comprise or consist of silica, polystyrene, metal, polymer, carbon particles or core-shell particles.

5. The method according to any preceding claim, wherein the particles are coated with functional groups or a polymeric or silica coating.

6. The method according to any preceding claim, wherein the particles comprise or consist of silica or silica coated iron oxide particles.

7. The method according to any preceding claim, wherein the particles are 1-500 nm in size.

8. The method according to any preceding claim, wherein the particles are incubated in the sample for a period of at least 2 minutes.

9. The method according to any preceding claim, wherein the protein corona are isolated from the sample and resuspended into a resuspension solution.

10. The method according to any preceding claim, wherein the method further comprises performing one or more wash steps on the protein corona following isolation.

11. The method according to any preceding claim, wherein the isolated protein corona is washed only once.

12. A method of glycoprotein profiling of a sample, wherein the glycoprotein profile of the sample is determined by enrichment of glycoproteins according to the method of any preceding claim.

13. The method according to claim 12, wherein the method of glycoprotein profiling further comprises a step of determining the glycan profile of the enriched glycoprotein(s).

14. A method of glycan profiling of a glycoprotein in a sample, wherein the glycan profile is determined by enrichment of the glycoprotein according to the method of any one of claims 1-11 and determining the glycan content of the glycoprotein.

15. The method according to any preceding claim, wherein the glycoprotein comprises or consists of fibrinogen and/or apolipoprotein (Apo) A1, or histidine rich glycoproteins.

16. A method of diagnosis of a disease or condition of a subject, the method comprising the enrichment of glycoproteins in a sample from the subject in accordance with any one of claims 1-11, wherein the glycoprotein and/or glycan profile of the protein corona is determined; and
wherein the glycoprotein and/or glycan profile is indicative of a disease or condition.

17. A method of monitoring the progression or remission of a disease or condition in a subject, the method comprising the enrichment of glycoproteins in a first sample from the subject in accordance with any one of claims 1-11, wherein the glycoprotein and/or glycan profile of the protein corona is determined and the glycoprotein and/or glycan profile is indicative of the state of the disease or condition; and
further comprising the enrichment of glycoproteins in a subsequent sample from the subject in accordance with the any one of claims 1-11, wherein the glycoprotein and/or glycan profile of the protein corona is determined and wherein a change in the determined glycoprotein and/or glycan profile between the first sample and subsequent sample is indicative of the progression or remission of the disease or condition.

18. The method according to claim 16 or 17, further comprising administering a treatment for the disease or condition.

19. A method of screening for biomarkers of a disease or condition, the method comprising the comparison of the glycoprotein and/or glycan profile of the protein corona from a sample of one or more subjects afflicted with the disease or condition relative to a control, such as the glycoprotein and/or glycan profile of the protein corona from a sample of one or more subjects that are not afflicted with the disease or condition;
wherein a difference in glycoprotein and/or glycan profile is indicative of a biomarker for the disease or condition; and wherein the method comprises the enrichment of glycoproteins in the sample from the subject in accordance with any one of claims 1-11.

20. A method of treatment for cancer in a subject, the method comprising:
obtaining or having obtained results of a cancer diagnosis for the subject in accordance with the method of claim 16; and
administering a treatment for the cancer to the subject.

21. A method of diagnosis for lung cancer, the method comprising the detection of one or more lung cancer biomarkers in a sample from a subject, wherein the biomarkers are selected from fibrinogen-derived glycan FA2G2S1 and FA3G3S2;
wherein a decrease in the level FA2G2S1 and FA3G3S2 is indicative of lung cancer, optionally wherein the lung cancer is non-small cell lung cancer.

22. The method according to claim 21, wherein the presence or level of the biomarkers is determined in accordance with the method according to any of claims 1-15.

23. A composition comprising a sample and a protein corona suspension within the sample, wherein the protein corona is formed around particles, such as silica particles, at a protein to particle surface area ratio of 11-330 mg/m² or 660 to 1760 mg/m².
